(19) United States
(12) Patent Application Publication
(10) Pub. No.: US 2015/0080651 A1
(43) Pub. Date: Mar. 19, 2015

(54) ENDOSCOPE APPARATUS

(71) Applicant: OLYMPUS CORPORATION, Tokyo (JP)

(72) Inventor: Motoo Azuma, Tokorozawa-shi (JP)

(73) Assignee: OLYMPUS CORPORATION, Tokyo (JP)

(21) Appl. No.: 14/547,936

(22) Filed: Nov. 19, 2014

Related U.S. Application Data
(63) Continuation of application No. PCT/JP2013/064827, filed on May 29, 2013.

(30) Foreign Application Priority Data
May 31, 2012 (JP) 2012-125120

(51) Int. Cl. A61B 1/045; A61B 1/00; A61B 1/05

(52) U.S. Cl. CPC A61B 1/045; A61B 1/05; A61B 1/00188; A61B 1/00009
USPC 600/103

(57) ABSTRACT

An endoscope apparatus includes an illumination unit, an optical system including an optical lens and a diaphragm unit configured to adjust a size of an opening, an imaging unit including a solid state imaging device and configured to output an image signal, a region setting unit configured to set a region of interest in an image formed by the image signal, an estimation value calculation unit configured to calculate and output an estimation value showing a focus level in the region of interest, an estimation value storage unit configured to store the estimation value, an estimation value comparison unit configured to compare a reference value and the estimation value and outputs the comparison result, and a diaphragm control unit configured to output a control signal for controlling the opening of the diaphragm unit based on the comparison result.

This application is a continuation application based on a PCT Application No. PCT/JP2013/064827, filed May 29, 2013, whose priority is claimed on Japanese Patent Application No. 2012-125120, filed May 31, 2012. The contents of both the PCT Application and the Japanese Application are incorporated herein by reference.

### BACKGROUND OF THE INVENTION

1. Field of the Invention

The present invention relates to focal depth control in an electronic endoscope apparatus which has a high-definition solid state imaging device having fine pixels.

2. Description of Related Art

In an endoscope system, a state in which a depth of field of a photographed image is large and the whole photographed image is focused on, i.e., a so-called pan-focus state in which an object is focused on from a short distance to a long distance is ideal. For this reason, an imaging unit of the endoscope is preferably controlled in a state in which a diaphragm is narrowed as much as possible.

In an electronic endoscope using a solid state imaging device, in order to respond to requirements of high image quality of the photographed image, the number of pixels in the solid state imaging device is being increased. Since space in a distal end section of the endoscope is limited, the pixel structure of the solid state imaging device is miniaturized. However, according to the miniaturization of the pixel structure, problems related to a decrease in sensitivity of the solid state imaging device and darkening of the photographed image may occur. When a diameter of a permissible circle of confusion is reduced, a shallow focal depth may also occur as an optical problem.

For example, in an endoscope apparatus disclosed in Japanese Unexamined Patent Application, First Publication No. H07-299029, a method of reconciling both appropriate brightness and a maximum focal depth by controlling an opening of a diaphragm unit based on brightness information obtained from the solid state imaging device is disclosed. More specifically, when the brightness information obtained from the solid state imaging device is larger than predetermined brightness, the size of the opening of the diaphragm unit is controlled to be primarily reduced. Meanwhile, when the brightness information obtained from the solid state imaging device is smaller than the predetermined brightness, a light source apparatus is driven such that a quantity of light is primarily increased in a state in which the size of the opening of the diaphragm unit is minimized. After the quantity of light from the light source apparatus is maximized, the size of the opening of the diaphragm unit is controlled to be increased. According to the above-mentioned control, in the endoscope apparatus disclosed in Japanese Unexamined Patent Application, First Publication No. H07-299029, the appropriate brightness and the maximum focal depth can be reconciled. In Japanese Unexamined Patent Application, First Publication No. H07-299029, a method of controlling a focal position, based on the brightness information obtained by detecting the brightness of the imaged object image, as an optical path length between the optical system and the solid state imaging device is varied, is also disclosed.

### SUMMARY OF THE INVENTION

According to a first aspect of the present invention, an endoscope apparatus includes: an illumination unit configured to radiate light from a light source toward an object; an optical system including an optical lens configured to form an object image, and a diaphragm unit configured to adjust a size of an opening in a plurality of steps; an imaging unit including a solid state imaging device configured to convert an optical image of the object captured through the optical system into an electric signal in accordance with the optical image, the imaging unit being configured to output an image signal in accordance with an image formed based on the electric signal; a region setting unit configured to set at least one region of interest in the image formed by the image signal output from the imaging unit; an estimation value calculation unit configured to calculate an estimation value showing a focus level in the region of interest and output the estimation value; an estimation value storage unit configured to store the estimation value output from the estimation value calculation unit; an estimation value comparison unit configured to read the previous estimation value stored in the estimation value storage unit as a reference value, compare the reference value and the current estimation value output from the estimation value calculation unit, and output a comparison result of the reference value and the estimation value; and a diaphragm control unit configured to output a control signal for controlling the opening of the diaphragm unit based on the comparison result output from the estimation value comparison unit.

According to a second aspect of the present invention, in the endoscope apparatus according to the first aspect of the present invention, the imaging unit may further include a gain adjustment unit configured to adjust brightness of the image formed based on the electric signal by electrically adjusting the electric signal of each of frames output from the solid state imaging device. The imaging unit may output the image signal in accordance with the electric signal of each of the frames adjusted such that the image has predetermined brightness.

According to a third aspect of the present invention, in the endoscope apparatus according to the first aspect of the present invention, the illumination unit may include a light adjustment unit configured to adjust a quantity of light radiated by the light source. The imaging unit may adjust the quantity of light radiated by the light source by the illumination unit such that brightness of the image formed based on the electric signal at each of frames output from the solid state imaging device becomes predetermined brightness.

According to a fourth aspect of the present invention, in the endoscope apparatus according to the first aspect of the present invention, the illumination unit may include a light adjustment unit configured to adjust a quantity of light radiated by the light source. The imaging unit may further include a gain adjustment unit configured to adjust brightness of the image formed based on the electric signal by electrically adjusting the electric signal at each of frames output from the solid state imaging device. The imaging unit may adjust, by the gain adjustment unit, the electric signal of the frame output from the solid state imaging device after the quantity of light radiated by the light source is maximized by the illumination unit.

According to a fifth aspect of the present invention, in the endoscope apparatus according to the first aspect of the present invention, the region setting unit may set the plurality of regions of interest to the entire image formed by the image signal output from the imaging unit with no gap.

According to a sixth aspect of the present invention, in the endoscope apparatus according to the first aspect of the present invention, the region setting unit may discretely set the plurality of regions of interest to the image formed by the image signal output from the imaging unit with gaps.

According to a seventh aspect of the present invention, in the endoscope apparatus according to the first aspect of the present invention, the region setting unit may set the regions of interest with equal sizes.

According to an eighth aspect of the present invention, in the endoscope apparatus according to the first aspect of the present invention, the illumination unit may include a light adjustment unit configured to adjust a quantity of light radiated by the light source. The imaging unit may further include a gain adjustment unit configured to adjust brightness of the image formed based on the electric signal by electrically adjusting the electric signal at each of frames output from the solid state imaging device. The imaging unit may adjust the quantity of light radiated by the light source by the illumination unit, and output the image signal of each of the frames obtained by adjusting, by the gain adjustment unit, the electric signal of each of the frames output from the solid state imaging device with the light radiated by the light source having the adjusted quantity of light radiated by the light source. The estimation value calculation unit may calculate the estimation value corresponding to the image signal of each of the frames, based on the image signal of each of the frames obtained by adjusting the quantity of light radiated by the light source and electrically adjusting the electric signal by the imaging unit.

According to a ninth aspect of the present invention, in the endoscope apparatus according to the eighth aspect of the present invention, the estimation value calculation unit may calculate the estimation value in a current frame at each of the regions of interest set by the region setting unit. The estimation value storage unit may store the estimation value in the current frame output from the estimation value calculation unit at each of the regions of interest. The estimation value comparison unit may compare the estimation value of each of the regions of interest in the current frame output from the estimation value calculation unit and the reference value corresponding to each of the regions of interest in a previous one frame read from the estimation value storage unit, and output the comparison result corresponding to each of the regions of interest obtained by comparing the estimation value and the reference value. The diaphragm control unit may control the opening of the diaphragm unit to be driven by at least one step in the same direction as a direction in which the opening of the diaphragm unit has been controlled when moved from the previous one frame to the current frame, if a number of the comparison results representing that the estimation value in the current frame is larger than the reference value in the previous one frame is equal to or larger than a preset number in the comparison results corresponding to each of the regions of interest output from the estimation value comparison unit. The diaphragm control unit may control the opening of the diaphragm unit to be driven by at least one step in an opposite direction to the direction in which the opening of the diaphragm unit has been controlled when moved from the previous one frame to the current frame, if the number of the comparison results representing that the estimation value in the current frame is larger than the reference value in the previous one frame is smaller than the preset number.

According to a tenth aspect of the present invention, in the endoscope apparatus according to the eighth aspect of the present invention, the estimation value calculation unit may calculate a total value obtained by adding the each estimation value corresponding to each of the regions of interest set by the region setting unit, and output the total value as the estimation value in a current frame. The estimation value storage unit may store the estimation value in the current frame output from the estimation value calculation unit. The estimation value comparison unit may compare the estimation value in the current frame output from the estimation value calculation unit and the reference value in a previous one frame read from the estimation value storage unit, and output the comparison result obtained by comparing the estimation value and the reference value. The diaphragm control unit may control the opening of the diaphragm unit to be driven by at least one step in the same direction as a direction in which the opening of the diaphragm unit has been controlled when moved from the previous one frame to the current frame, if the comparison result output from the estimation value comparison unit represents that the estimation value serving as the total value in the current frame is larger than the reference value serving as the total value in the previous one frame. The diaphragm control unit may control the opening of the diaphragm unit to be driven by at least one step in an opposite direction to the direction in which the opening of the diaphragm unit has been controlled when moved from the previous one frame to the current frame, if the comparison result represents that the estimation value serving as the total value in the current frame is equal to or smaller than the reference value serving as the total value in the previous one frame.

According to an eleventh aspect of the present invention, in the endoscope apparatus according to the eighth aspect of the present invention, the estimation value calculation unit may calculate a weighted average value obtained through weighted average of the each estimation value corresponding to each of the regions of interest set by the region setting unit, and output the weighted average value as the estimation value in a current frame. The estimation value storage unit may store the estimation value in the current frame output from the estimation value calculation unit. The estimation value comparison unit may compare the estimation value in the current frame output from the estimation value calculation unit and the reference value in a previous one frame read from the estimation value storage unit, and output the comparison result obtained by comparing the estimation value and the reference value. The diaphragm control unit may control the opening of the diaphragm unit to be driven by at least one step in the same direction as a direction in which the opening of the diaphragm unit has been controlled when moved from a previous one frame to the current frame, if the comparison result output from the estimation value comparison unit represents that the estimation value serving as the weighted average value in the current frame is larger than the reference value serving as the weighted average value in the previous one frame. The diaphragm control unit may control the opening of the diaphragm unit to be driven by at least one step in an opposite direction to the direction in which the opening of the diaphragm unit has been controlled when moved from the previous one frame to the current frame, if the comparison result represents that the estimation value serving as the weighted average value in the current frame is equal to or smaller than the reference value serving as the weighted average value in the previous one frame.

According to a twelfth aspect of the present invention, in the endoscope apparatus according to the first aspect of the present invention, the diaphragm control unit may control the opening of the diaphragm unit to move to a position at which the estimation value is maximized if a maximum estimation value is detected from the each estimation value in the current frame corresponding to each of the regions of interest set by the region setting unit, control the opening of the diaphragm unit such that a state of the opening of the diaphragm unit at the position is held for a preset time, and control the opening of the diaphragm unit to be driven by at least one step in a direction in which the opening of the diaphragm unit is reduced after the preset time elapses.

According to a thirteenth aspect of the present invention, in the endoscope apparatus according to the twelfth aspect of the present invention, the estimation value comparison unit may calculate an absolute value of a difference between the estimation value of the current frame corresponding to each of the regions of interest set by the region setting unit and the corresponding reference value in a previous one frame stored in the estimation value storage unit, and output the calculated result of the absolute value as a calculation result of each of the regions of interest. The diaphragm control unit may terminate holding the state of the opening of the diaphragm unit for the preset time and restart control of the opening of the diaphragm unit from a next frame, if the at least one calculation result in the calculation result corresponding to each of the regions of interest output from the estimation value comparison unit during the preset time exceeds a preset value.

According to a fourteenth aspect of the present invention, in the endoscope apparatus according to the eighth aspect of the present invention, the optical system may further include a lens driving unit configured to be interlocked with the opening of the diaphragm unit and to set a focal position of the optical lens.

According to a fifteenth aspect of the present invention, in the endoscope apparatus according to the fourteenth aspect of the present invention, the lens driving unit may set the focal position of the optical lens such that, when the opening of the diaphragm unit is largest, the object disposed at a proximal end is included in a focus range of the optical system and the object disposed at the proximal end to the object disposed at a distal end are gradually included in the focus range of the optical system as the opening of the diaphragm unit is reduced gradually, and when the opening of the diaphragm unit is smallest, a range in which the optical image of the object is converted into the electric signal by the solid state imaging device is entirely included in the focus range of the optical system.

### BRIEF DESCRIPTION OF THE DRAWINGS

FIG. 1 is a block diagram showing an example of a schematic configuration of an endoscope system according to a first embodiment of the present invention.

FIG. 2 is a timing chart showing an example of a diaphragm control operation in the endoscope system according to the first embodiment of the present invention.

FIG. 3 is a view schematically showing an example of a region of interest, in which an estimation value is calculated, in a solid state imaging device included in the endoscope system according to the first embodiment of the present invention.

FIG. 4 is a view schematically showing an example of the entire diaphragm control operation in the endoscope system according to the first embodiment of the present invention.

FIG. 5A is a view schematically showing another example of the region of interest, in which an estimation value is calculated, in the solid state imaging device included in the endoscope system according to the first embodiment of the present invention.

FIG. 5B is a view schematically showing still another example of the region of interest, in which an estimation value is calculated, in the solid state imaging device included in the endoscope system according to the first embodiment of the present invention.

FIG. 5C is a view schematically showing still another example of the region of interest, in which an estimation value is calculated, in the solid state imaging device included in the endoscope system according to the first embodiment of the present invention.

FIG. 5D is a view schematically showing still another example of the region of interest, in which an estimation value is calculated, in the solid state imaging device included in the endoscope system according to the first embodiment of the present invention.

FIG. 6 is a block diagram showing an example of a schematic configuration of an endoscope system according to a second embodiment of the present invention.

FIG. 7 is a timing chart showing an example of a diaphragm control operation in the endoscope system according to the second embodiment of the present invention.

FIG. 8 is a block diagram showing an example of a schematic configuration of an endoscope system according to a third embodiment of the present invention.

FIG. 9 is a timing chart showing an example of a diaphragm control operation in the endoscope system according to the third embodiment of the present invention.

FIG. 10 is a view schematically showing an example of the entire diaphragm control operation in the endoscope system according to the third embodiment of the present invention.

FIG. 11 is a block diagram showing an example of a schematic configuration of an endoscope system according to a fourth embodiment of the present invention.

FIG. 12 is a timing chart showing an example of a diaphragm control operation in the endoscope system according to the fourth embodiment of the present invention.

FIG. 13 is a view schematically showing an example of the entire diaphragm control operation in the endoscope system according to the fourth embodiment of the present invention.

### DETAILED DESCRIPTION OF THE INVENTION

### First Embodiment

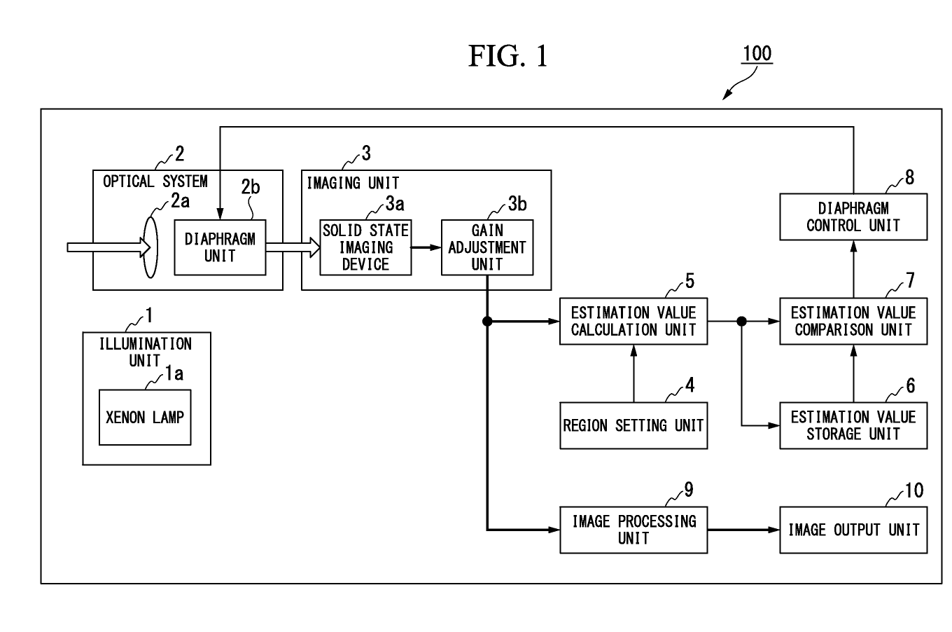

Hereinafter, an endoscope apparatus according to an embodiment of the present invention will be described with reference to the accompanying drawings. FIG. 1 is a block diagram showing an example of a schematic configuration of an endoscope system according to the first embodiment. An endoscope system 100 shown in FIG. 1 includes an illumination unit 1, an optical system 2, an imaging unit 3, a region setting unit 4, an estimation value calculation unit 5, an estimation value storage unit 6, an estimation value comparison unit 7, and a diaphragm control unit 8. The endoscope system 100 may further include an image processing unit 9 and an image output unit 10.

The illumination unit 1 includes a xenon lamp 1a serving as a light source apparatus. The illumination unit 1 radiates light from the xenon lamp 1a to an object in a body photographed by the endoscope system 100. The optical system 2 includes an optical lens 2a configured to form an object image, and a diaphragm unit 2b configured to adjust a size of an opening according to a control signal output from the diaphragm control unit 8. The optical system 2 delivers object light to the imaging unit 3.

The imaging unit 3 includes a solid state imaging device 3a and a gain adjustment unit 3b. The solid state imaging device 3a photoelectrically converts an optical image of the object captured through the optical system 2, and converts the optical image into an electric signal of each frame. The gain adjustment unit 3b adjusts intensity of the electric signal output from the solid state imaging device 3a to an appropriate level according to a state of light and shade of the entire image imaged by the solid state imaging device 3a. The imaging unit 3 outputs the electric signal of each frame having the level adjusted by the gain adjustment unit 3b to the estimation value calculation unit 5 and the image processing unit 9 as the image signal.

The region setting unit 4 sets regions of interest by dividing the entire image of one frame output by the imaging unit 3 into, for example, a plurality of regions having the same size with no gap. A method of dividing the regions of interest by the region setting unit 4 will be described below in detail.

The estimation value calculation unit 5 detects an amount of a high frequency element, from which a noise element is removed, from the image signal after level adjustment input from the imaging unit 3 at every region of interest set by the region setting unit 4. The estimation value calculation unit 5 calculates an estimation value corresponding to the amount of the high frequency element of each of the detected regions of interest. The estimation value calculation unit 5 outputs the calculated estimation value to the estimation value storage unit 6 and the estimation value comparison unit 7. The estimation value is a value showing the degree of focus in the region of interest.

The estimation value storage unit 6 individually stores the estimation value of each of the regions of interest input from the estimation value calculation unit 5 to one frame. The estimation value storage unit 6 outputs each of the stored estimation values to the estimation value comparison unit 7 as a reference value.

The estimation value comparison unit 7 compares magnitudes of the estimation value of each of the regions of interest input from the estimation value calculation unit 5 and the reference value of the corresponding region of interest stored in the estimation value storage unit 6 at each of the regions of interest set by the region setting unit 4. The estimation value comparison unit 7 outputs the result of comparing the magnitudes (hereinafter referred to as “a comparison result”) to the diaphragm control unit 8.

Comparison of the estimation value and the reference value by the estimation value comparison unit 7 is performed by reading the reference value of the corresponding region of interest from the estimation value storage unit 6, i.e., the estimation value of the previous frame, at the timing at which the estimation value calculation unit 5 outputs the estimation value of the current frame. The estimation value comparison unit 7 outputs a signal representing whether or not the estimation value input from the estimation value calculation unit 5 is larger than the reference value read from the estimation value storage unit 6 to the diaphragm control unit 8 as a comparison result.

The diaphragm control unit 8 determines whether the driving control is performed in a direction of reducing the opening of the diaphragm unit 2b included in the optical system 2 (hereinafter referred to as “a diaphragm direction”) or a direction of increasing the opening (hereinafter referred to as “an opening direction”) based on the comparison result of the entire region of interest input from the estimation value comparison unit 7. More specifically, the diaphragm control unit 8 counts the number of regions of interest, at which it is determined that “the estimation value>the reference value” in the comparison result input from the estimation value comparison unit 7. When the counted number is equal to or larger than a preset number, the diaphragm control unit 8 determines that the diaphragm unit 2b is driven by one step in the same direction in which it was previously driven (one of the diaphragm direction and the opening direction). Here, the diaphragm control unit 8 outputs a control signal for controlling the driving of the diaphragm unit 2b in the determined direction. When the counted number is smaller than the preset number, the diaphragm control unit 8 determines that the diaphragm unit 2b is driven by one step in an opposite direction to that in which it was previously driven. Here, the diaphragm control unit 8 outputs a control signal for controlling the driving of the diaphragm unit 2b in the determined direction.

The image processing unit 9 performs image processing through which the image signal after level adjustment in each of the frames input from the imaging unit 3 is converted into, for example, a format displayed in a monitor connected to the endoscope system 100. The image processing unit 9 outputs the image signal passing through the image processing (hereinafter referred to as “image data”) to the image output unit 10. The image output unit 10 outputs and displays the image data input from the image processing unit 9 to, for example, the monitor connected to the endoscope system 100 at each of the frames.

According to the above-mentioned configuration, in the endoscope system 100, the entire image of one frame imaged by the solid state imaging device 3a is divided into a plurality of regions of interest. Based on the estimation values of the divided regions of interest, the opening of the diaphragm unit 2b when an image of the next frame is imaged is controlled. That is, in the endoscope system 100, as the opening of the diaphragm unit 2b is controlled based on a distance at which the objects are distributed, focused images are obtained in all the imaged images. Accordingly, in the endoscope system 100, when all the imaged objects are distributed in a range of a short distance in the vicinity of the focus, the opening of the diaphragm unit 2b can be increased, and a high resolution image can be photographed. In the endoscope system 100, when not all the imaged objects are provided in the vicinity of the focus or when all the objects are distributed at a range of a long distance, as the opening of the diaphragm unit 2b is reduced, like the related art, the image in the pan-focus state in which the image is focused on as a whole (hereinafter referred to as “a focused image”) can be photographed.

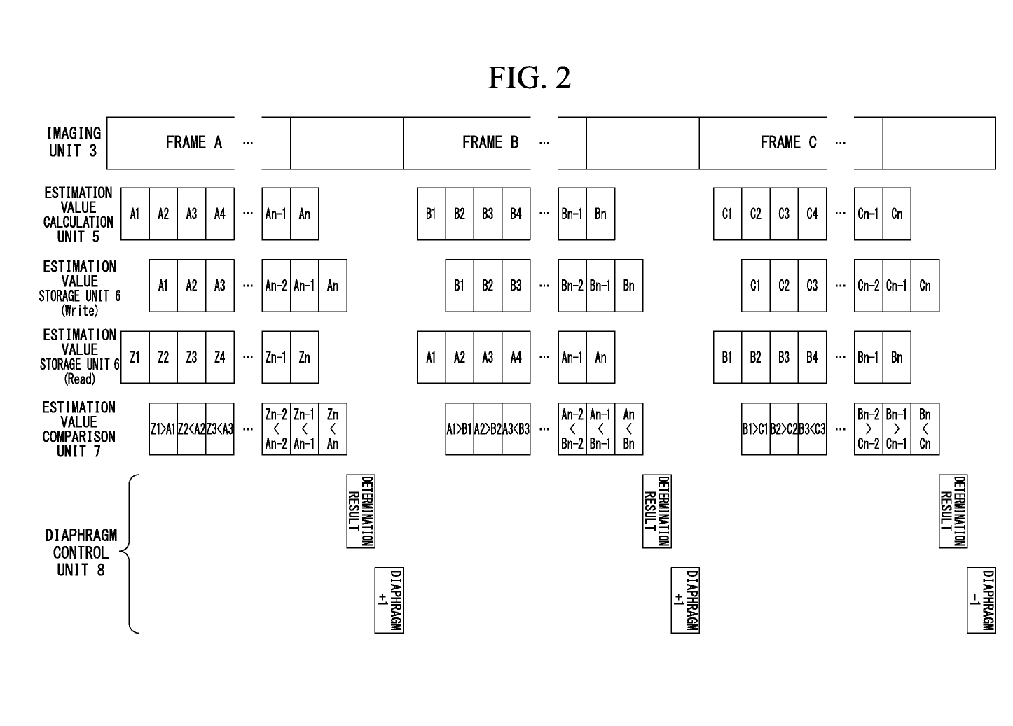

Next, an operation of the endoscope system 100 according to the first embodiment will be described. FIG. 2 is a timing chart showing an example of a diaphragm control operation in the endoscope system 100 according to the first embodiment. As described above, in the endoscope system 100, the estimation value calculation unit 5 calculates the estimation value of the region of interest at each of the frames imaged by the solid state imaging device 3a. The endoscope system 100 controls the driving of the diaphragm unit 2b based on the estimation values of the regions of interest.

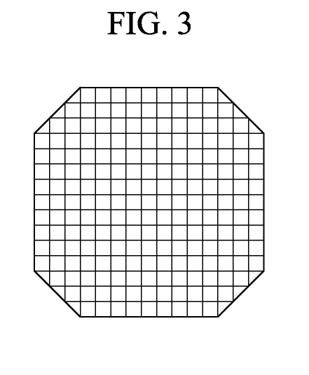

In the following description, as shown in FIG. 3, the case in which the region setting unit 4 sets the regions of interest by dividing the entire image of one frame output from the imaging unit 3 into a plurality of regions having the same size with no gap will be described. As the regions of interest are set as shown in FIG. 3, the driving of the diaphragm unit 2b can be controlled while the region of the observation target in the endoscope system 100 is set as the entire image.

The imaging unit 3 outputs an image signal according to the electric signal obtained by photoelectrically converting an optical image of the object of the current frame A captured through the optical system 2 to the estimation value calculation unit 5. In the endoscope system 100 according to the first embodiment, the imaging unit 3 includes the gain adjustment unit 3b configured to correct the brightness of the image, which is imaged by the solid state imaging device 3a, varying depending on the opening and closing of the diaphragm unit 2b to an appropriate brightness. When the image of the frame A imaged by the solid state imaging device 3a is dark, the imaging unit 3 amplifies the electric signal output from the solid state imaging device 3a using the gain adjustment unit 3b. Accordingly, the imaging unit 3 outputs the image signal having a level adjusted to the certain brightness, regardless of the opening and closing of the diaphragm unit 2b, to the estimation value calculation unit 5 as the image signal of the frame A.

The estimation value calculation unit 5 calculates the estimation value obtained by detecting the amount of the high frequency element, from which the noise element is removed, from the image signal input from the imaging unit 3 at each of the regions of interest set by the region setting unit 4 and obtained by equally dividing the entire image of the frame A into n regions with no gap. In estimation values A1 to An shown in FIG. 2, “A” represents the estimation value of the frame A, and “1 to n” represent the corresponding regions of interest. In the following description, when the estimation value of the frame A is represented with no distinction of the regions of interest, it is referred to as “an estimation value A”. The estimation value calculation unit 5 sequentially outputs the calculated estimation values A1 to An corresponding to the regions of interest of the frame A to the estimation value storage unit 6 and the estimation value comparison unit 7.

The calculation of the estimation values by the estimation value calculation unit 5 can be performed by a known technology using, for example, a bandpass filter or the like. Since the calculation method of the estimation value is a method generally used, for example, when a function of auto-focus of a digital camera or the like is realized, a detailed description thereof will be omitted here.

The estimation value storage unit 6 sequentially stores the estimation values A1 to An of the frame A input from the estimation value calculation unit 5 into the storage regions of the estimation value storage unit 6 corresponding to the regions of interest.

The estimation value comparison unit 7 compares the estimation value A of the frame A input from the estimation value calculation unit 5 and a reference value Z serving as the estimation value corresponding to the same region of interest of the previous frame Z stored in the estimation value storage unit 6. In reference values Z1 to Zn shown in FIG. 2, “Z” represents a reference value of the frame Z, and “1 to n” represent the corresponding regions of interest. In the following description, when the reference value of the frame Z is represented with no distinction of the regions of interest, it is referred to as “the reference value Z.”

More specifically, as shown in FIG. 2, the estimation value comparison unit 7 sequentially reads the previous reference values Z1 to Zn of the frame Z of the corresponding regions of interest stored in the estimation value storage unit 6 at timings at which the estimation value calculation unit 5 outputs each of the estimation values A1 to An of the frame A. The estimation value comparison unit 7 sequentially compares magnitudes of the estimation value A and the reference value Z at each of the regions of interest. The estimation value comparison unit 7 sequentially outputs the comparison result representing whether or not the estimation value A is larger than the reference value Z to the diaphragm control unit 8.

Provisionally, the case in which the diaphragm unit 2b is driven by one step in the negative direction (the opening direction) from the previous frame Z to the current frame A will be assumed. The estimation value comparison unit 7 first outputs the comparison result obtained by comparing magnitude correlations of the reference value Z1 of an initial (first) region of interest and the estimation value A1 to the diaphragm control unit 8. FIG. 2 shows the case in which the magnitude comparison result of the reference value Z1 and the estimation value A1 is that “the reference value Z1>the estimation value A1.” Here, the estimation value comparison unit 7 outputs, for example, the signal representing that the magnitude comparison result (hereinafter referred to as “a comparison result signal”)=“0” to the diaphragm control unit 8 as the comparison result in the first region of interest.

Next, the estimation value comparison unit 7 compares the magnitude correlations of the reference value Z2 of the second region of interest and the estimation value A2. The estimation value comparison unit 7 outputs the comparison result in the second region of interest to the diaphragm control unit 8. FIG. 2 shows the case in which the magnitude comparison result of the reference value Z2 and the estimation value A2 is that “the reference value Z2<the estimation value A2.” Here, the estimation value comparison unit 7 outputs, for example, the comparison result signal=“1” to the diaphragm control unit 8 as the comparison result in the second region of interest.

Hereinafter, similarly, the estimation value comparison unit 7 repeats comparisons of the magnitude correlations of the reference values Z and the estimation values A of the regions of interest. The estimation value comparison unit 7 sequentially outputs the comparison result signal=“1” when “the estimation value A>the reference value Z” is true and the comparison result signal=“0” when false to the diaphragm control unit 8 as the comparison results in the regions of interest. In this way, the estimation value comparison unit 7 sequentially outputs the comparison results obtained by comparing the magnitude correlations of the reference values Z and the estimation values A with respect to all of n regions of interest to the diaphragm control unit 8.

The diaphragm control unit 8 counts the number of regions of interest in which it is determined that the estimation value A of the frame A is larger than the reference value Z of the frame Z, i.e., “the estimation value A>the reference value Z,” in the comparison results sequentially input from the estimation value comparison unit 7. For example, in FIG. 2, the number of the regions of interest in which the comparison result signal=“1” is counted. The diaphragm control unit 8 determines a direction in which the diaphragm unit 2b is driven based on the counted result and the preset constant.

More specifically, if the counted result is equal to or larger than a preset constant M, the diaphragm control unit 8 determines that the current frame A has a larger number of focused regions of interest than the previous frame Z. Here, the diaphragm control unit 8 controls the diaphragm unit 2b to be driven by one step in the same direction in which it was previously driven. On the other hand, if the counted result is smaller than the preset constant M, the diaphragm control unit 8 determines that the current frame A has a smaller number of focused regions of interest than the previous frame Z. Here, the diaphragm control unit 8 controls the diaphragm unit 2b to be driven by one step in an opposite direction to that in which it was previously driven. In FIG. 2, as a result of determination in the current frame A, the case in which the diaphragm unit 2b is controlled to be driven by one step in the positive direction (the diaphragm direction) is shown.

Subsequently, the imaging unit 3 outputs the image signal according to the electric signal obtained by photoelectrically converting the optical image of the object of the next frame B captured by the optical system 2 to the estimation value calculation unit 5. The estimation value calculation unit 5 calculates estimation values B1 to Bn corresponding to the regions of interest in the frame B. The estimation value calculation unit 5 sequentially outputs the estimation values B1 to Bn to the estimation value storage unit 6 and the estimation value comparison unit 7.

The estimation value comparison unit 7 replaces the reference values Z1 to Zn of the above-mentioned frame Z with the estimation values A1 to An of the frame A stored in the estimation value storage unit 6 in the previous frame A, respectively. The estimation value comparison unit 7 compares the reference value A with the estimation value B of the frame B input from the estimation value calculation unit 5. The estimation value comparison unit 7 outputs the comparison result signal corresponding to each of the regions of interest to the diaphragm control unit 8. Accordingly, the diaphragm control unit 8 controls driving of the diaphragm unit 2b based on the determination result in the frame B. FIG. 2 shows the case in which the diaphragm unit 2b is controlled to be further driven by one step in the positive direction (the diaphragm direction) based on the determination result in the frame B, and further, the diaphragm unit 2b is controlled to be driven by one step in the negative direction (the opening direction) based on the determination result in the next frame C.

In this way, in the endoscope system 100, as the estimation values in the region of interest are calculated and the calculated estimation value and the estimation value (the reference value) of the previous frame are compared at each frame imaged by the solid state imaging device 3a, the opening of the diaphragm unit 2b in the next frame is controlled. Accordingly, in the endoscope system 100, the opening of the diaphragm unit 2b, i.e., the range in which the object is focused on, follows a variation in the distance at which the object is distributed, and the focused image focused on the entire image can be photographed.

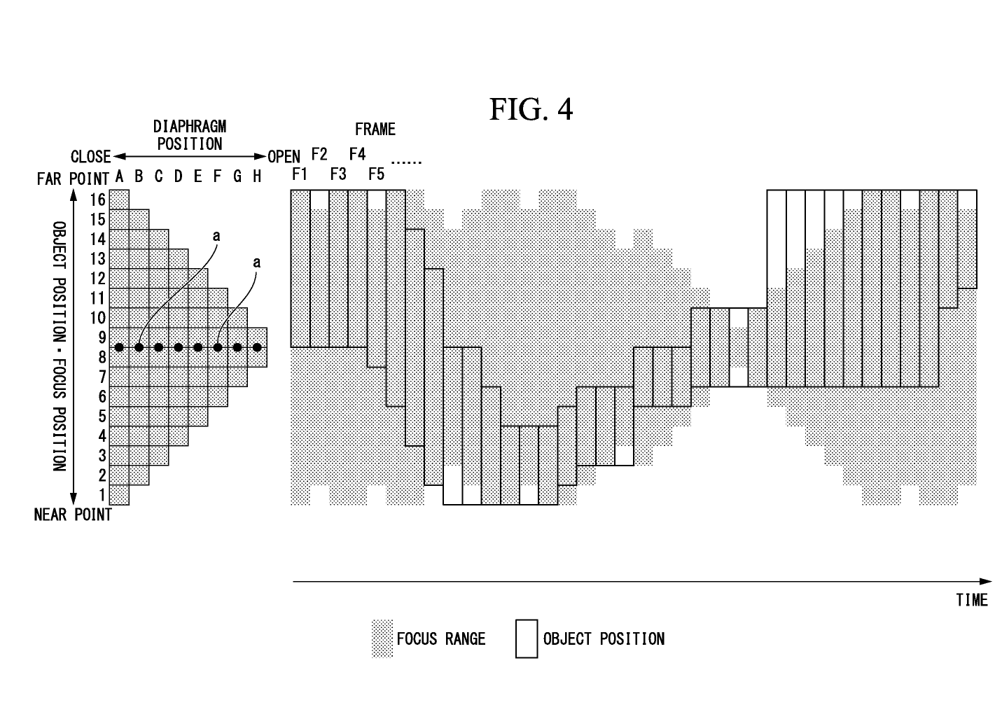

The entire control operation of the diaphragm unit 2b in the endoscope system 100 according to the first embodiment will be described. FIG. 4 is a view schematically showing an example of the entire diaphragm control operation in the endoscope system 100 according to the first embodiment. FIG. 4 schematically shows a relation between the diaphragm position and the focus range in the optical system 2 in which a focal position is set (fixed) to a central position of the distance to the object serving as the photographing target and the entire range of the object position is focused on when the diaphragm unit is maximally narrowed. In addition, FIG. 4 schematically shows the relation between the object position and the focus range in the frames when the moving object is photographed using the optical system 2 as time elapses.

A variation in focus range by the control of the diaphragm unit 2b in the endoscope system 100 will be described using FIG. 4. The object position shown in FIG. 4 is a position of the object in a depth direction. The focus range shown in FIG. 4 is a range of focusing in the depth direction.

A relation between the diaphragm position and the focus range in the optical system 2 of the endoscope system 100 will be described. The optical system 2 can control eight diaphragm positions A to H shown in FIG. 4. Focus ranges at the diaphragm positions are ranges shown in FIG. 4. More specifically, the diaphragm position A, i.e., the focus range when the size of the opening of the diaphragm unit 2b is minimized, is a range of object positions 1 to 16, i.e., from a near point closest to the distance to the object to a far point farthest from the distance to the object. The focus range at the diaphragm position B is a range of the object positions 2 to 15. The focus range at the diaphragm position C is a range of the object positions 3 to 14. The focus range at the diaphragm position D is a range of the object positions 4 to 13. The focus range at the diaphragm position E is a range of the object positions 5 to 12. The focus range at the diaphragm position F is a range of the object positions 6 to 11. The focus range at the diaphragm position G is a range of the object positions 7 to 10. The diaphragm position H, i.e., the focus range when the size of the opening of the diaphragm unit 2b is maximized, is a range of the object positions 8 to 9.

As described above, the focal position of the optical system 2 is fixed to a position of a center of a distance to the object serving as the photographing target, i.e., a position of a black spot a of a center of the focus range in each of the diaphragm positions.

In the endoscope system 100, the image of each frame is photographed using the optical system 2. Here, in the endoscope system 100, according to the above-mentioned diaphragm control operation, the driving control of the diaphragm unit 2b is performed at each frame, and the diaphragm position when the image of the next frame is photographed is varied.

FIG. 4 shows the case in which the object disposed at the range of the object position (the range of the position of the object in the depth direction) shown by a thick-bordered box in a frame F1 is photographed using the opening of the diaphragm unit 2b as the diaphragm position A, and based on the determination result of the diaphragm control in the frame F1, the opening of the diaphragm unit 2b upon photographing of a frame F2 is controlled to be driven to the diaphragm position B by one step in the negative direction (the opening direction). Next, the case in which the object disposed at the range of the object position shown by the thick-bordered box in the frame F2 is photographed at the diaphragm position B of the opening of the diaphragm unit 2b, and based on the determination result of the diaphragm control in the frame F2, the opening of the diaphragm unit 2b upon photographing of a frame F3 is controlled to be driven to the diaphragm position A by one step in the positive direction (the diaphragm direction) is shown. Next, the case in which the object disposed at the range of the object position shown by the thick-bordered box in the frame F3 is photographed at the diaphragm position A of the opening of the diaphragm unit 2b, and based on the determination result of the diaphragm control in the frame F3, the opening of the diaphragm unit 2b upon photographing of a frame F4 is at the diaphragm position A with no modification is shown. Next, the case in which the object disposed at the range of the object position shown by the thick-bordered box in the frame F4 is photographed at the diaphragm position A of the opening of the diaphragm unit 2b, and based on the determination result of the diaphragm control in the frame F4, the opening of the diaphragm unit 2b upon photographing of a frame F5 is controlled to be driven to the diaphragm position B by one step in the negative direction (the opening direction) is shown.

Similarly, based on the determination result of the diaphragm control in each of the frames, the diaphragm unit 2b is controlled to be driven at the diaphragm position at which the next frame is photographed. Accordingly, as shown in FIG. 4, the diaphragm position can be controlled to follow the object position.

In the endoscope system 100, as described above, the focal position of the optical system 2 is fixed to a position of the black spot a of the center of the focus range. For this reason, in the endoscope system 100, the object position is controlled to be included in the focus range. That is, while the focused image cannot be photographed normally when the object is in the vicinity of the near point or the far point, a depth of field is increased by controlling the opening of the diaphragm unit 2b in the diaphragm direction according to the diaphragm control operation in the endoscope system 100. Accordingly, in the endoscope system 100, as shown in FIG. 4, even when the object position is deviated toward the near point or the far point, the focused image in which the object is included within the focus range can be photographed.

As described above, in the endoscope system 100 according to the first embodiment, the entire image of each of the frames is divided into the plurality of regions of interest. Based on the estimation values of the divided regions of interest, the opening of the diaphragm unit 2b when the image of the next frame is imaged is controlled to follow a variation in the distance at which the object is distributed. Accordingly, the high resolution image can be photographed at the appropriate depth of field according to the distance to the object without excessively increasing the depth of field and decreasing the resolution of the photographed image.

In the endoscope system 100 according to the first embodiment, the case in which the diaphragm unit 2b is driven by one step at each of the frames has been described. However, the number of steps in which the diaphragm unit 2b is controlled to be driven is not limited to one step. For example, the driving control of the diaphragm unit 2b may be appropriately varied, such as controlling driving the diaphragm unit 2b in a plurality of steps, rather than one step, or not operating the diaphragm unit 2b, according to the number of regions of interest in which it is determined that the estimation value of the current frame is larger than the reference value of the previous frame, i.e., “the estimation value>the reference value.” For example, the driving control of the diaphragm unit 2b may be appropriately varied according to a difference between the reference value of the previous frame and the estimation value of the current frame when the estimation value comparison unit 7 compares the magnitude correlations.

In the endoscope system 100 according to the first embodiment, the case in which the estimation value comparison unit 7 compares the reference value of the previous frame and the estimation value of the current frame at each of the regions of interest has been described. However, a comparison method of the reference value and the estimation value in the estimation value comparison unit 7 is not limited to the above-mentioned comparison method. For example, the estimation value comparison unit 7 may incorporate the estimation values of all the regions of interest into one value by calculating a statistical value, and compare the incorporated reference value and the incorporated estimation values.

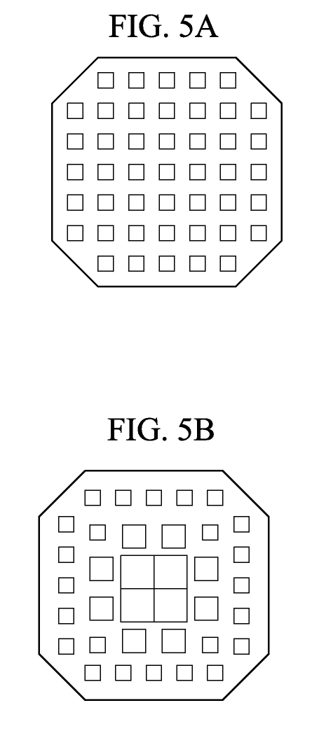

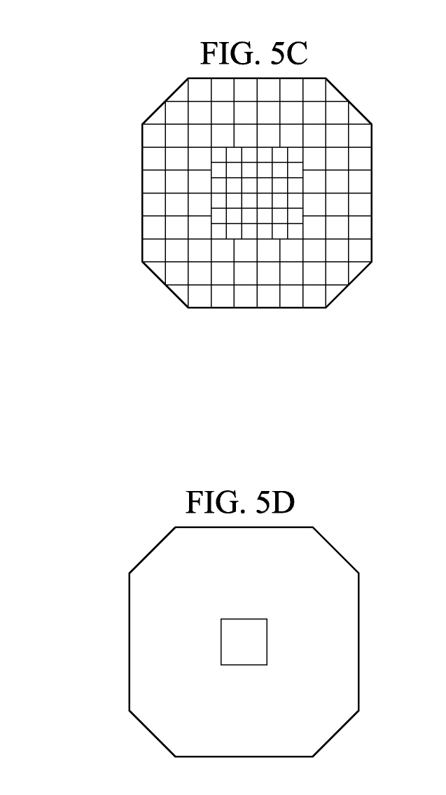

In the endoscope system 100 according to the first embodiment, the case in which the regions of interest set by the region setting unit 4 are regions having equal sizes with no gap as shown in FIG. 3 has been described. However, the regions of interest set by the region setting unit 4 is not limited to the above-mentioned example. For example, various regions shown in FIGS. 5A to 5D may be set as the regions of interest. FIG. 5A shows an example of a region of interest divided into a plurality of discrete regions having gaps in an image of one frame. FIG. 5B shows an example of a region of interest divided into regions having irregular sizes that increase toward a center of the image when the region is divided into a plurality of discrete regions having gaps in an image of one frame. FIG. 5C shows an example of a region of interest divided into regions having irregular sizes that reduce toward a center of an image when the region is divided into a plurality of regions with no gap in the entire image of one frame. FIG. 5D shows an example of a region of interest as one region at a center of an image of one frame. In the endoscope system 100 according to the first embodiment, the estimation value calculation unit 5 calculates the estimation value at each of the regions of interest set by the region setting unit 4. The estimation value comparison unit 7 compares the reference value of the previous frame and the estimation value of the current frame at each of the regions of interest. Accordingly, the region setting unit 4 sets the same region of interest with at least two frames without setting different regions of interest at each frame.

In the endoscope system 100 according to the first embodiment, the case in which the light source apparatus included in the illumination unit 1 is the xenon lamp 1a has been described. However, the light source apparatus is not limited to the xenon lamp 1a. A halogen lamp, a Light Emitting Diode (LED), a laser, or the like, may be installed at the illumination unit 1 as the light source apparatus. In addition, a light adjustment unit may be installed in the illumination unit 1.

### Second Embodiment

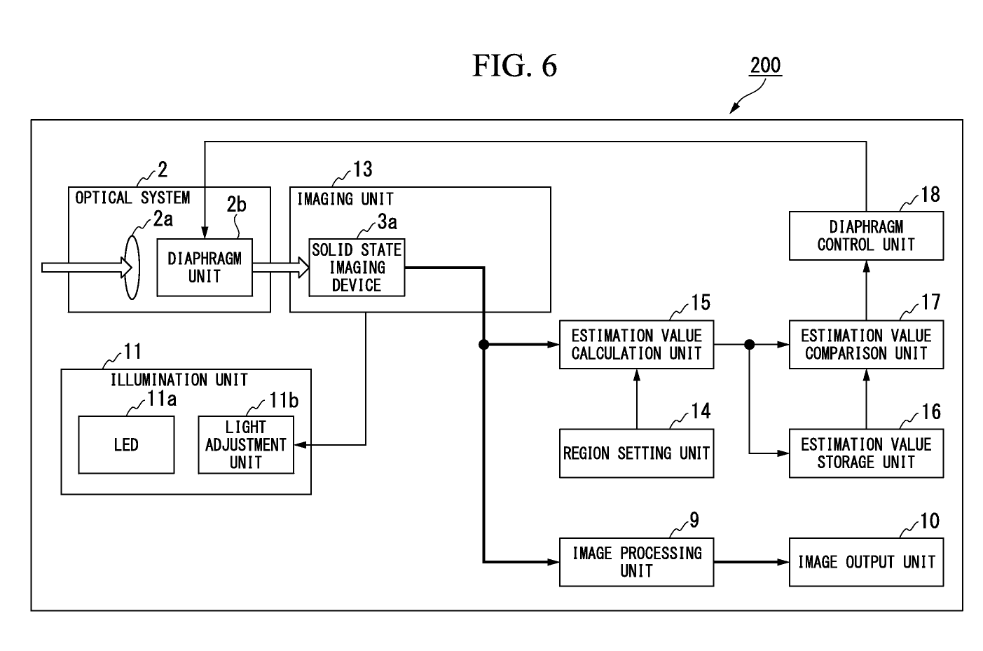

Next, an endoscope system according to a second embodiment of the present invention will be described. FIG. 6 is a block diagram showing an example of a schematic configuration of the endoscope system according to the second embodiment. An endoscope system 200 shown in FIG. 6 includes an illumination unit 11, an optical system 2, an imaging unit 13, a region setting unit 14, an estimation value calculation unit 15, an estimation value storage unit 16, an estimation value comparison unit 17, and a diaphragm control unit 18. The endoscope system 200 may further include an image processing unit 9 and an image output unit 10. In the endoscope system 200 according to the second embodiment, the optical system 2, the image processing unit 9, and the image output unit 10 are the same components as in the endoscope system 100 according to the first embodiment. The components of the endoscope system 200 according to the second embodiment different from the components of the endoscope system 100 according to the first embodiment may also include components having the same configuration as the endoscope system 100 according to the first embodiment. Accordingly, the same components and the same configurations as the endoscope system 100 according to the first embodiment will be designated by the same reference numerals, and a detailed description thereof will be omitted here.

The illumination unit 11 includes an LED 11a as a light source apparatus. The illumination unit 11 further includes a light adjustment unit 11b configured to adjust light emitted from the LED 11a. The illumination unit 11 radiates the light from the LED 11a modulated by the light adjustment unit 11b to an object in a body photographed by the endoscope system 200.

The imaging unit 13 has a configuration in which the gain adjustment unit 3b is removed from the imaging unit 3 installed in the endoscope system 100 according to the first embodiment. That is, the imaging unit 13 includes only the solid state imaging device 3a in the imaging unit 3 installed in the endoscope system 100 according to the first embodiment. The imaging unit 13 outputs an electric signal of each frame obtained by photoelectrically converting the optical image of the object captured by the solid state imaging device 3a through the optical system 2 to the estimation value calculation unit 15 and the image processing unit 9 as the image signal.

The region setting unit 14 sets the region of interest in which the image of one frame output from the imaging unit 13 is divided into, for example, a plurality of discrete regions disposed with gaps. Here, the region setting unit 14 sets the size of each of the regions of interest to an irregular size that increases toward a center of the image. The size of the region of interest set by the region setting unit 14 is determined, for example, to match a distortion aberration property of the optical lens 2a installed in the optical system 2.

The estimation value calculation unit 15 detects an amount of a high frequency element, from which a noise element is removed, from the image signal input from the imaging unit 13 at each region of interest set by the region setting unit 14. The estimation value calculation unit 15 calculates the estimation value at each of the regions of interest based on the detected amount of the high frequency element at each of the regions of interest. The estimation value calculation unit 15 outputs the one estimation value obtained by adding the estimation values to the estimation value storage unit 16 and the estimation value comparison unit 17.

The estimation value storage unit 16 stores the one estimation value input from the estimation value calculation unit 15. The estimation value storage unit 16 outputs the stored one estimation value to the estimation value comparison unit 17 as the reference value. The estimation value storage unit 16 can reduce a circuit scale because only the one estimation value is stored.

The estimation value comparison unit 17 reads the reference value serving as the one estimation value of the previous frame (the summed estimation value) from the estimation value storage unit 16 at the timing at which the estimation value calculation unit 15 outputs the estimation value of the current frame. The estimation value comparison unit 17 compares magnitudes of the read one reference value and the one estimation value input from the estimation value calculation unit 15. The estimation value comparison unit 17 outputs one comparison result (comparison result signal) representing whether the one estimation value input from the estimation value calculation unit 15 is larger than the one reference value read from the estimation value storage unit 16 to the diaphragm control unit 18.

The diaphragm control unit 18 determines whether the opening of the diaphragm unit 2b is controlled to be driven in the diaphragm direction or the opening direction based on the one comparison result input from the estimation value comparison unit 17. More specifically, if the comparison result input from the estimation value comparison unit 17 is that “the estimation value>the reference value,” the diaphragm control unit 18 determines that the diaphragm unit 2b is driven by one step in the same direction in which it was previously driven (any one of the diaphragm direction and the opening direction). Here, the diaphragm control unit 18 outputs a control signal for controlling driving of the diaphragm unit 2b in the determined direction. If the comparison result input from the estimation value comparison unit 17 is not that “the estimation value>the reference value,” the diaphragm control unit 18 determines that the diaphragm unit 2b is driven by one step in an opposite direction to that in which it was previously driven. Here, the diaphragm control unit 18 outputs a control signal for controlling driving of the diaphragm unit 2b in the determined direction.

The image processing unit 9 outputs image-processed image data, in which the image signal of each of the frames input from the imaging unit 13 is converted into, for example, a format displayed on a monitor connected to the endoscope system 200, to the image output unit 10. The image output unit 10 outputs and displays the image data input from the image processing unit 9 to, for example, the monitor connected to the endoscope system 200 at each of the frames.

According to the above-mentioned configuration, in the endoscope system 200, the image of the one frame imaged by the solid state imaging device 3a is divided into the plurality of regions of interest. Based on the one estimation value calculated from the image signals of the divided regions of interest, the opening of the diaphragm unit 2b when the image of the next frame is imaged is controlled. Accordingly, in the endoscope system 200, like the endoscope system 100 according to the first embodiment, as the opening of the diaphragm unit 2b is controlled based on the distance at which the object is distributed, the focused image having high resolution photographed by increasing the opening of the diaphragm unit 2b, or the same focused image as the related art photographed by reducing the opening of the diaphragm unit 2b can be photographed.

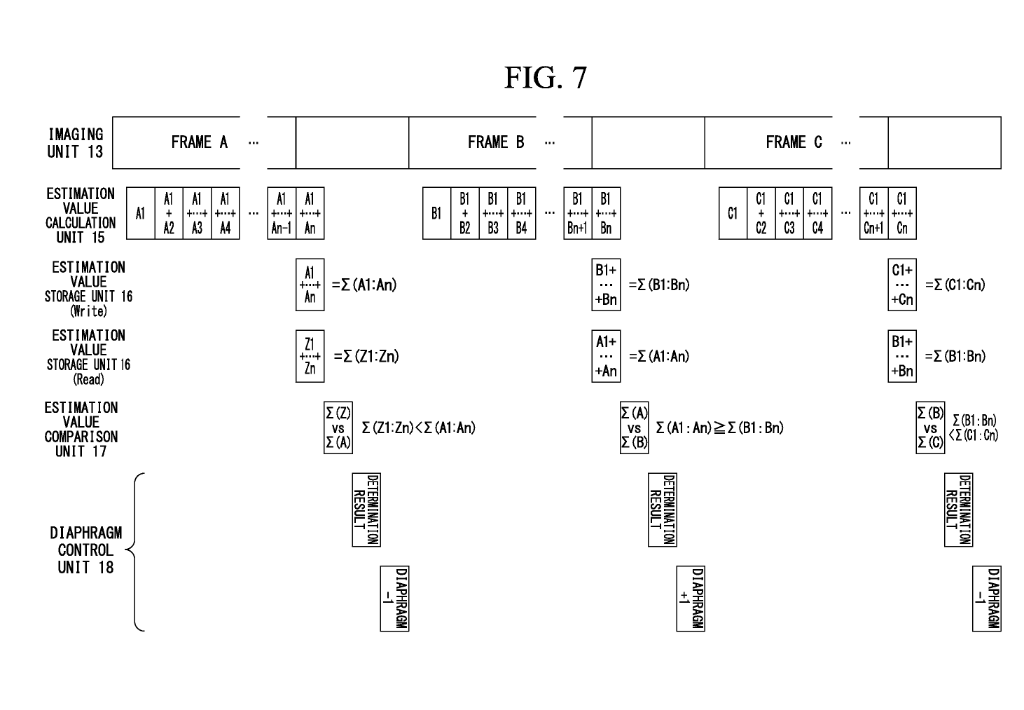

Next, an operation of the endoscope system 200 according to the second embodiment will be described. FIG. 7 is a timing chart showing an example of a diaphragm control operation in the endoscope system 200 according to the second embodiment. As described above, in the endoscope system 200, the estimation value calculation unit 15 calculates the estimation value of the region of interest at each of the frames imaged by the solid state imaging device 3a. The endoscope system 200 outputs the one estimation value obtained by adding the estimation values. The endoscope system 200 controls driving of the diaphragm unit 2b based on the one estimation value.

In the following description, the case in which the region setting unit 14 sets the region of interest obtained by dividing the image of the one frame output from the imaging unit 13 into the plurality of discrete regions disposed with gaps and having irregular sizes that increase toward a center of the image will be described. In this case, the region of interest set by the region setting unit 14 is, for example, a region of interest disposed as shown in FIG. 5B. As the region of interest is set as shown in FIG. 5B, the number of regions of interest in the endoscope system 200 is reduced, and a circuit scale when the estimation value calculation unit 15 calculates the estimation value of each of the regions of interest can be reduced.

The imaging unit 13 outputs the image signal according to the electric signal obtained by photoelectrically converting the optical image of the object of the current frame A captured through the optical system 2 to the estimation value calculation unit 15. In the endoscope system 200 according to the second embodiment, the light adjustment unit 11b configured to adjust light radiated by the LED 11a radiated to the object is installed in the illumination unit 11 as a means of correcting the brightness of the image, which is imaged by the solid state imaging device 3a, varying depending on the opening and closing of the diaphragm unit 2b to an appropriate brightness. The imaging unit 13 controls the light adjustment unit 11b in the illumination unit 11 and increases the emission intensity of the LED 11a when the image of the frame A imaged by the solid state imaging device 3a is dark. Accordingly, the imaging unit 13 outputs the image signal adjusted to the constant brightness regardless of the opening and closing of the diaphragm unit 2b to the estimation value calculation unit 15 as the image signal of the frame A.

The estimation value calculation unit 15 calculates the estimation value in which the amount of the high frequency element, from which a noise element is removed, is detected from the image signal input from the imaging unit 13, at each of the regions of interest set by the region setting unit 14 and obtained by dividing the image of the frame A into n regions discretely disposed with gaps having sizes that increase toward a center of the image. In “A” and “1 to n” of the estimation values A1 to An shown in FIG. 7, like the description of the operation of the endoscope system 100 according to the first embodiment, “A” represents the estimation value of the frame A and “1 to n” represent the corresponding regions of interest. The estimation value calculation unit 15 outputs the one estimation value Σ(A1:An) obtained by adding the calculated estimation values A1 to An corresponding to the regions of interest of the frame A to the estimation value storage unit 16 and the estimation value comparison unit 17.

The estimation value storage unit 16 stores the one estimation value Σ(A1:An) of the frame A input from the estimation value calculation unit 15.

The estimation value comparison unit 17 compares the one estimation value Σ(A1:An) of the frame A input from the estimation value calculation unit 15 and the one reference value Σ(Z1:Zn), which is the summed one estimation value of the previous frame Z stored in the estimation value storage unit 16. In “Z” and “1 to n” in the reference values Z1 to Zn shown in FIG. 7, like the description of the operation of the endoscope system 100 according to the first embodiment, “Z” represents the reference value of the frame Z, and “1 to n” represent the corresponding regions of interest.

More specifically, as shown in FIG. 7, the estimation value comparison unit 17 reads the one reference value Σ(Z1:Zn) of the frame Z stored in the estimation value storage unit 16 at the timing at which the estimation value calculation unit 15 outputs the one estimation value Σ(A1:An) of the frame A. The estimation value comparison unit 17 compares magnitudes of the estimation value Σ(A1:An) and the reference value Σ(Z1:Zn). The estimation value comparison unit 17 outputs the comparison result representing whether the estimation value Σ(A1:An) is larger than the reference value Σ(Z1:Zn) to the diaphragm control unit 18.

Provisionally, the case in which the diaphragm unit 2b is driven by one step in the negative direction (the opening direction) from the previous frame Z to the current frame A is supposed. The estimation value comparison unit 17 outputs the comparison result obtained by comparing the magnitude correlations of the reference value Σ(Z1:Zn) and the estimation value Σ(A1:An) to the diaphragm control unit 18. FIG. 7 shows the case in which the magnitude comparison result of the reference value Σ(Z1:Zn) and the estimation value Σ(A1:An) is that “the reference value Σ(Z1:Zn)<the estimation value Σ(A1:An).” Here, the estimation value comparison unit 17 outputs, for example, the comparison result signal=“1” representing the magnitude comparison result to the diaphragm control unit 18 as the comparison result in the frame A.

The diaphragm control unit 18 determines a direction in which the diaphragm unit 2b is driven based on the comparison result input from the estimation value comparison unit 17. The diaphragm control unit 18 controls driving of the diaphragm unit 2b in the determined direction. More specifically, if the comparison result input from the estimation value comparison unit 17 is that “the reference value Σ(Z1:Zn)<the estimation value Σ(A1:An),” i.e., the comparison result signal is “1,” the diaphragm control unit 18 determines that the current frame A has a larger number of focused regions of interest than the previous frame Z. Here, the diaphragm control unit 18 controls the diaphragm unit 2b to be driven by one step in the same direction as the direction in which the diaphragm unit 2b was previously driven. FIG. 7 shows the case in which the diaphragm unit 2b is controlled to be driven by one step in the negative direction (the opening direction), as a result of determination in the current frame A.

The imaging unit 13 outputs the image signal according to the electric signal obtained by photoelectrically converting the optical image of the object of the next frame B captured through the optical system 2 to the estimation value calculation unit 15. The estimation value calculation unit 15 calculates the estimation values B1 to Bn corresponding to the regions of interest in the frame B. The estimation value calculation unit 15 outputs the one estimation value Σ(B1:Bn) obtained by adding the estimation values B1 to Bn to the estimation value storage unit 16 and the estimation value comparison unit 17.

The estimation value comparison unit 17 replaces the above-mentioned one reference value Σ(Z1:Zn) with the one estimation value Σ(A1:An) of the frame A stored in the estimation value storage unit 16 in the previous frame A. The estimation value comparison unit 17 performs magnitude comparison of the reference value Σ(A1:An) and the one estimation value Σ(B1:Bn) of the frame B input from the estimation value calculation unit 15. The estimation value comparison unit 17 outputs the comparison result to the diaphragm control unit 18. FIG. 7 shows the case in which the magnitude comparison result of the reference value Σ(A1:An) and the estimation value Σ(B1:Bn) is that “the reference value Σ(A1:An)≧the estimation value Σ(B1:Bn).” Here, the estimation value comparison unit 17 outputs, for example, the comparison result signal=“0” representing the magnitude comparison result to the diaphragm control unit 18 as the comparison result in the frame B.

The diaphragm control unit 18 determines that the current frame B has a smaller number of focused regions of interest than the previous frame A if the comparison result input from the estimation value comparison unit 17 is that “the reference value Σ(A1:An)≧the estimation value Σ(B1:Bn),” i.e., the comparison result signal is “0.” Here, the diaphragm control unit 18 controls the diaphragm unit 2b to be driven by one step in an opposite direction to the direction in which the diaphragm unit 2b was previously driven. FIG. 7 shows the case in which the diaphragm unit 2b is controlled to be driven by one step in the positive direction (the diaphragm direction), as a result of determination in the current frame B.

Thereafter, similarly, the diaphragm unit 2b is controlled to be driven based on the determination result in the next frame. FIG. 7 shows the case in which the diaphragm unit 2b is controlled to be driven by one step in the negative direction (the opening direction) based on the determination result in the frame C.

In this way, in the endoscope system 200, the estimation value of each of the regions of interest is calculated at each of the frames imaged by the solid state imaging device 3a. As the one estimation value obtained by adding the calculated estimation values and the one estimation value (reference value) of the previous frame are compared, the opening of the diaphragm unit 2b in the next frame is controlled. Accordingly, in the endoscope system 200, like the endoscope system 100 according to the first embodiment, the focused image in which the opening of the diaphragm unit 2b, i.e., the range in which the object is focused on, follows a variation in the distance at which the object is distributed can be photographed.

The entire control operation of the diaphragm unit 2b in the endoscope system 200 according to the second embodiment is the same as that of the diaphragm unit 2b in the endoscope system 100 according to the first embodiment shown in FIG. 4, and thus a detailed description thereof will be omitted here.

As described above, in the endoscope system 200 according to the second embodiment, the entire image of each of the frames is divided into a plurality of regions of interest. Based on the one estimation value obtained by adding the estimation values of the divided regions of interest, the opening of the diaphragm unit 2b when the image of the next frame is imaged is controlled to follow a variation in the distance at which the object is distributed. Accordingly, like the endoscope system 100 according to the first embodiment, a high resolution image can be photographed at an appropriate depth of field according to the distance to the object without excessively increasing the depth of field and decreasing the resolution of the photographed image.

In the endoscope system 200 according to the second embodiment, the case in which the diaphragm unit 2b is driven by one step at each of the frames has been described. However, the number of steps in which the diaphragm unit 2b is controlled to be driven is not limited to the above-mentioned example. For example, in consideration of the magnitudes of the reference value Σ(Z1:Zn) and the estimation value Σ(A1:An), the driving control of the diaphragm unit 2b may be appropriately varied, such as control of non-operation of the diaphragm unit 2b or control of driving of the diaphragm unit 2b in a plurality of steps.

In the endoscope system 200 according to the second embodiment, the case in which the estimation value calculation unit 15 calculates the one estimation value obtained by adding the estimation values of the regions of interest has been described. However, a method of calculating the one estimation value is not limited to the above-mentioned example. The estimation value calculation unit 15 may calculate the one estimation value through a method other than summation of the estimation values of the regions of interest. For example, like the endoscope system 100 according to the first embodiment, the estimation value calculation unit 15 may output the calculated estimation values of the regions of interest. The estimation value comparison unit 17 may compare the reference value of the previous frame and the estimation value of the current frame at each of the regions of interest.

In the endoscope system 200 according to the second embodiment, the case in which the regions of interest set by the region setting unit 14 are a plurality of regions discretely disposed with gaps and having sizes that increase toward the center of the image has been described. However, the regions of interest set by the region setting unit 14 are not limited to the above-mentioned example. For example, like the endoscope system 100 according to the first embodiment, various regions such as disposition as shown in FIG. 3 or FIGS. 5A to 5D may be set as the region of interest.

In the endoscope system 200 according to the second embodiment, the case in which the light source apparatus installed in the illumination unit 11 is the LED 11a and the light adjustment unit 11b configured to adjust the light emitted from the LED 11a is included has been described. However, the light source apparatus may be a halogen lamp, a xenon lamp, a laser, or the like. The light adjustment unit 11b is not installed in the illumination unit 11, and alternatively, a gain adjustment unit configured to correct the brightness of the image, which is imaged by the solid state imaging device 3a, varying depending on the opening and closing of the diaphragm unit 2b to an appropriate brightness may be installed in the imaging unit 13.

### Third Embodiment

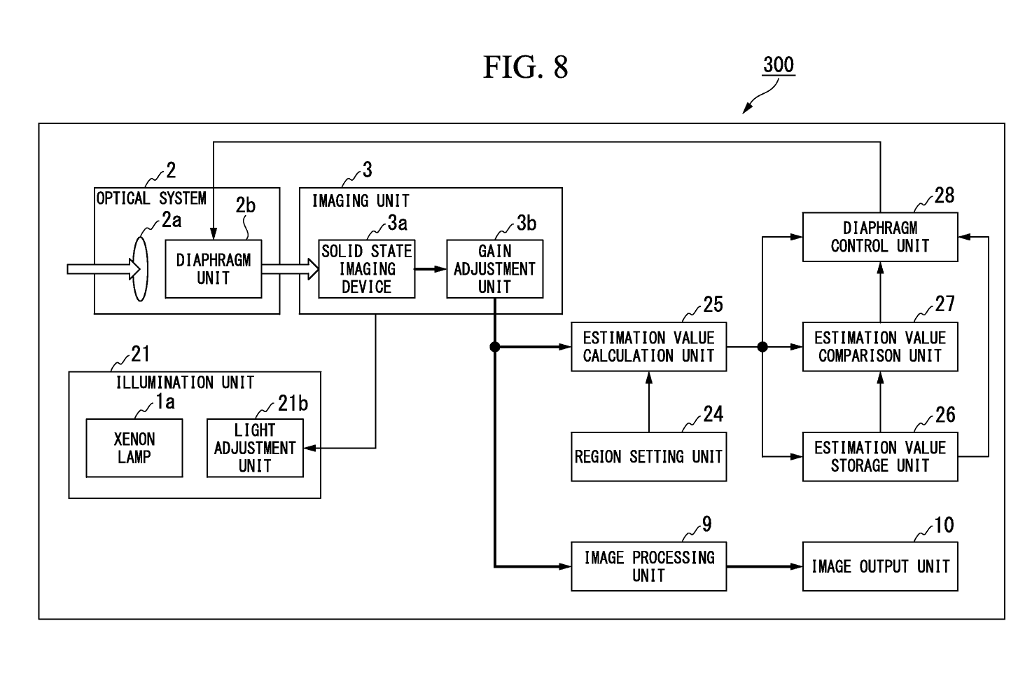

Next, an endoscope system according to a third embodiment of the present invention will be described. FIG. 8 is a block diagram showing an example of a schematic configuration of the endoscope system according to the third embodiment. An endoscope system 300 shown in FIG. 8 includes an illumination unit 21, an optical system 2, an imaging unit 3, a region setting unit 24, an estimation value calculation unit 25, an estimation value storage unit 26, an estimation value comparison unit 27, and a diaphragm control unit 28. The endoscope system 300 may further include an image processing unit 9 and an image output unit 10. In the endoscope system 300 according to the third embodiment, the optical system 2, the imaging unit 3, the image processing unit 9, and the image output unit 10 are the same components as in the endoscope system 100 according to the first embodiment. The components of the endoscope system 300 according to the third embodiment different from the components of the endoscope system 100 according to the first embodiment may also include components having the same configuration as the endoscope system 100 according to the first embodiment. Accordingly, the same components and the same configurations as the endoscope system 100 according to the first embodiment will be designated by the same reference numerals, and a detailed description thereof will be omitted here.

The illumination unit 21 includes a xenon lamp 1a serving as a light source apparatus. The illumination unit 21 further includes a light adjustment unit 21b configured to adjust a quantity of the light emitted from the xenon lamp 1a. The illumination unit 21 radiates the light from the xenon lamp 1a, a quantity of which is adjusted by the light adjustment unit 21b, to the object in a body photographed by the endoscope system 300.

The imaging unit 3 is the same as that of the endoscope system 100 according to the first embodiment. The imaging unit 3 adjusts a level of the electric signal of each of the frames imaged by the solid state imaging device 3a by the gain adjustment unit 3b. The imaging unit 3 outputs the adjusted electric signal to the estimation value calculation unit 25 and the image processing unit 9 as the image signal.

The region setting unit 24 sets, for example, region of interests divided into a plurality of discrete regions disposed with gaps in the image of the one frame output from the imaging unit 3. Here, the region setting unit 24 sets the regions of interest to have equal sizes.

The estimation value calculation unit 25 detects an amount of the high frequency element, from which a noise element is removed, from the image signal after level adjustment input from the imaging unit 3 at each of the regions of interest set by the region setting unit 24. The estimation value calculation unit 25 calculates the estimation value corresponding to the detected amount of the high frequency element of each of the regions of interest. The estimation value calculation unit 25 outputs the one estimation value obtained by performing a weighted average of the estimation values to the estimation value storage unit 26, the estimation value comparison unit 27, and the diaphragm control unit 28.

The estimation value storage unit 26 stores the one estimation value input from the estimation value calculation unit 25. The estimation value storage unit 26 outputs the stored one estimation value to the estimation value comparison unit 27 as a reference value. The estimation value storage unit 26 stores the estimation value corresponding to the plurality of frames required for detecting a position of the diaphragm unit 2b at which the estimation value becomes a maximum value (hereinafter referred to as “a maximum estimation value”) by the diaphragm control unit 28. The estimation value storage unit 26 outputs the stored estimation value corresponding to the plurality of frames to the diaphragm control unit 28.

The estimation value comparison unit 27 reads the reference value, which is the one estimation value of the previous one frame (the weight-averaged estimation value), from the estimation value storage unit 26 at the timing at which the estimation value calculation unit 25 outputs the estimation value of the current frame. The estimation value comparison unit 27 compares magnitudes of the read one reference value and the one estimation value input from the estimation value calculation unit 25. The estimation value comparison unit 27 outputs the one comparison result (comparison result signal) representing whether the one estimation value input from the estimation value calculation unit 25 is larger than the one reference value read from the estimation value storage unit 26 to the diaphragm control unit 28.

The diaphragm control unit 28 determines whether the opening of the diaphragm unit 2b is controlled to be driven in the diaphragm direction or the opening direction based on the one comparison result input from the estimation value comparison unit 27. More specifically, the diaphragm control unit 28 determines that the diaphragm unit 2b is driven by one step in the same direction in which it was previously driven (any one of the diaphragm direction and the opening direction) if the comparison result input from the estimation value comparison unit 27 is that “the estimation value>the reference value.” Here, the diaphragm control unit 28 outputs a control signal for controlling driving of the diaphragm unit 2b in the determined direction. The diaphragm control unit 28 determines that the diaphragm unit 2b is driven by one step in an opposite direction to that in which it was previously driven if the comparison result input from the estimation value comparison unit 27 is not “the estimation value>the reference value.” Here, the diaphragm control unit 28 outputs a control signal for controlling driving of the diaphragm unit 2b in the determined direction.

The diaphragm control unit 28 detects a maximum estimation value based on the estimation value corresponding to the plurality of frames input from the estimation value storage unit 26 and the estimation value of the current frame input from the estimation value calculation unit 25. A detection method of the maximum estimation value by the diaphragm control unit 28 is the same method as so-called climbing control widely used as a contrast type auto-focus operation in, for example, a digital camera or the like. For this reason, a detailed description of the detection method of the maximum estimation value by the diaphragm control unit 28 will be omitted here.

The diaphragm control unit 28 controls driving of the opening of the diaphragm unit 2b to reach the diaphragm position at which the estimation value is maximized when the maximum estimation value is detected. After that, the diaphragm control unit 28 pauses the driving control of the diaphragm unit 2b for a preset certain time T. Accordingly, the opening of the diaphragm unit 2b is held at the same diaphragm position for the certain time T. For this reason, a so-called wobbling operation performed after focusing at the diaphragm position at which the estimation value is maximized and moved (driven) forward and rearward in an optical axis direction of the optical lens 2a can be suppressed. In addition, the image quality of moving images by the endoscope system 300 can be improved.

In the endoscope system 300, even in a period of the certain time T while the diaphragm position is held, the estimation value calculation unit 25 calculates the estimation value, and continues to update the estimation value stored in the estimation value storage unit 26. The diaphragm control unit 28 drives the diaphragm unit 2b by one step in the diaphragm direction after the certain time T elapses. The diaphragm control unit 28 restarts the driving control of the diaphragm unit 2b based on the one comparison result input from the estimation value comparison unit 27.

The driving control of the diaphragm unit 2b by the diaphragm control unit 28 after the certain time T elapses is not limited to the one step in the above-mentioned diaphragm direction. The driving control of the diaphragm unit 2b may be in a plurality of steps or in the opening direction. Calculation of the estimation value by the estimation value calculation unit 25 and update of the estimation value in the estimation value storage unit 26 may be paused for the certain time T in which the diaphragm position is held.

The image processing unit 9 outputs the image data obtained through the image processing of converting the image signal of each of the frames input from the imaging unit 3 into, for example, a format displayed on a monitor connected to the endoscope system 300, to the image output unit 10. The image output unit 10 outputs and displays the image data input from the image processing unit 9 to, for example, the monitor connected to the endoscope system 300 at each of the frames.

According to the above-mentioned configuration, in the endoscope system 300, the image of the one frame imaged by the solid state imaging device 3a is divided into a plurality of regions of interest. Based on the one estimation value calculated from the image signal of the divided regions of interest, the opening of the diaphragm unit 2b when the image of the next frame is imaged is controlled. Accordingly, in the endoscope system 300, like the endoscope system 100 according to the first embodiment or the endoscope system 200 according to the second embodiment, as the opening of the diaphragm unit 2b is controlled based on the distance at which the object is distributed, the focused image having high resolution photographed by increasing the opening of the diaphragm unit 2b, or the same focused image as the related art photographed by reducing the opening of the diaphragm unit 2b can be photographed.

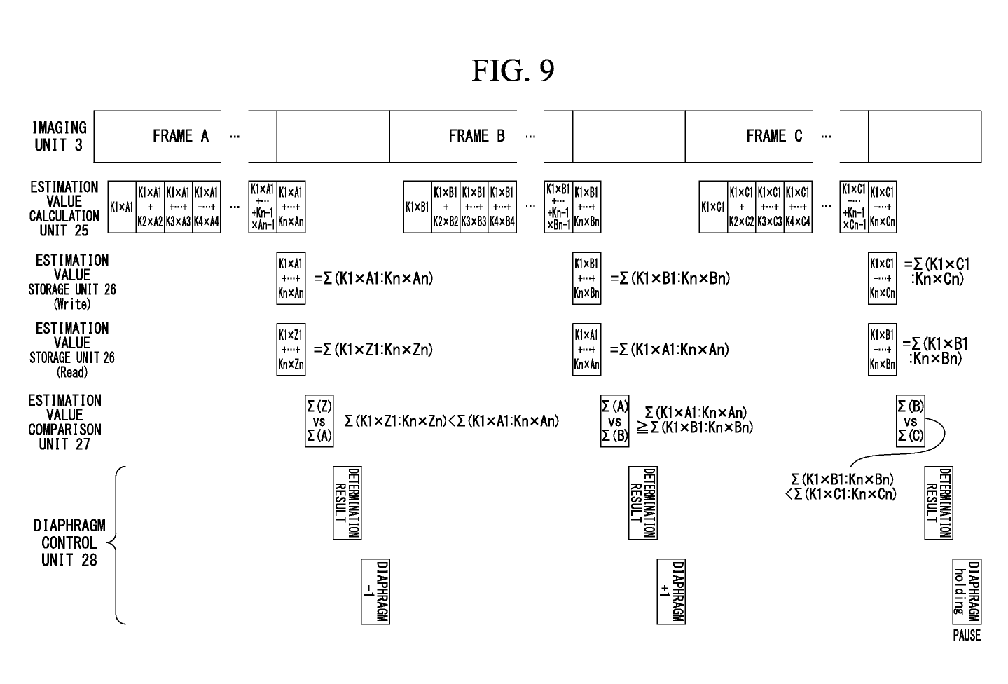

Next, an operation of the endoscope system 300 according to the third embodiment will be described. FIG. 9 is a timing chart showing an example of a diaphragm control operation in the endoscope system 300 according to the third embodiment. As described above, in the endoscope system 300, the estimation value calculation unit 25 calculates the estimation value of the region of interest at each of the frames imaged by the solid state imaging device 3a. The endoscope system 300 outputs the one estimation value obtained through the weighted average of the estimation values. The endoscope system 300 controls driving of the diaphragm unit 2b based on the one estimation value.

In the following description, the case in which the region setting unit 24 sets the regions of interest obtained by dividing the image of the one frame output from the imaging unit 3 into the plurality of regions discretely disposed with gaps and having equal sizes will be described. In this case, the regions of interest set by the region setting unit 24 are, for example, regions of interest disposed as shown in FIG. 5A. As the regions of interest are set as shown in FIG. 5A, the number of regions of interest in the endoscope system 300 is reduced, and a circuit scale when the estimation value calculation unit 25 calculates the estimation value of each of the regions of interest can be reduced. In addition, correction of the estimation value due to a difference in size of the regions of interest is unnecessary, and the circuit scale can be further reduced.

The imaging unit 3 outputs the image signal according to the electric signal obtained by photoelectrically converting the optical image of the object of the current frame A captured through the optical system 2 to the estimation value calculation unit 25. In the endoscope system 300 according to the third embodiment, as a means for correcting the brightness of the image, which is imaged by the solid state imaging device 3a, varying depending on the opening and closing of the diaphragm unit 2b to an appropriate brightness, the light adjustment unit 21b is installed in the illumination unit 21 and the gain adjustment unit 3b is installed in the imaging unit 3. When the image of the frame A imaged by the solid state imaging device 3a is dark, first, the imaging unit 3 primarily controls the light adjustment unit 21b in the illumination unit 21 and corrects the brightness such that the image of the frame A is brightened. After the light radiated by the illumination unit 21 is maximally brightened, the imaging unit 3 controls amplification of the electric signal output from the solid state imaging device 3a using the gain adjustment unit 3b, and thus the noise element of the image of the frame A is reduced. Accordingly, the imaging unit 3 outputs the image signal controlled to the certain brightness regardless of the opening and closing of the diaphragm unit 2b to the estimation value calculation unit 25 as the image signal of the frame A.

Here, the estimation value calculation unit 25 calculates the estimation value obtained by detecting the amount of the high frequency element, from which the noise element is removed, from the image signal input from the imaging unit 3 at each of the regions of interest set by the region setting unit 24 and obtained by dividing the image of the frame A into n regions discretely disposed with gaps and having equal sizes. In “A” and “1 to n” in the estimation values A1 to An shown in FIG. 9, like the description of the operation of the endoscope system 200 according to the second embodiment, “A” represents the estimation value of the frame A and “1 to n” represent the corresponding regions of interest. K1 to Kn shown in FIG. 9 are weighting coefficients applied to the estimation values A1 to An according to positions of the region of interest. The estimation value calculation unit 25 calculates the weighted average by multiplying the estimation values A1 to An corresponding to the regions of interest of the calculated frame A by the weighting coefficients K1 to Kn according to the positions of the regions of interest. The estimation value calculation unit 25 outputs the weighted average value to the estimation value storage unit 26, the estimation value comparison unit 27, and the diaphragm control unit 28 as one estimation value Σ(K1×A1:Kn×An).

In consideration of a property of symmetry in the image of the frame output from the imaging unit 3, calculation of the weighted average can be simplified by setting the weighting coefficients K1 to Kn to be vertically or horizontally symmetric.

The estimation value storage unit 26 stores the one estimation value Σ(K1×A1:Kn×An) of the frame A input from the estimation value calculation unit 25.

The estimation value comparison unit 27 compares the one estimation value Σ(K1×A1:Kn×An) of the frame A input from the estimation value calculation unit 25 and a reference value Σ(K1×Z1:Kn×Zn), which is one weighted average estimation value of the previous frame Z stored in the estimation value storage unit 26. In “Z” and “1 to n” in the reference values Z1 to Zn shown in FIG. 9, like the description of the operation of the endoscope system 200 according to the second embodiment, “Z” represents the reference value of the frame Z and “1 to n” represent the corresponding regions of interest.

More specifically, as shown in FIG. 9, the estimation value comparison unit 27 reads the one reference value Σ(K1×Z1:Kn×Zn) of the frame Z stored in the estimation value storage unit 26 at the timing at which the estimation value calculation unit 25 outputs the one estimation value Σ(K1×A1:Kn×An) of the frame A. The estimation value comparison unit 27 compares magnitudes of the estimation value Σ(K1×A1:Kn×An) and the reference value Σ(K1×Z1:Kn×Zn). The estimation value comparison unit 27 outputs the comparison result representing whether the estimation value Σ(K1×A1:Kn×An) is larger than the reference value Σ(K1×Z1:Kn×Zn) to the diaphragm control unit 28.

Provisionally, the case in which the diaphragm unit 2b is driven by one step in the negative direction (the opening direction) from the previous frame Z to the current frame A is supposed. The estimation value comparison unit 27 outputs the comparison result obtained by comparing the magnitude correlations of the reference value Σ(K1×Z1:Kn×Zn) and the estimation value Σ(K1×A1:Kn×An) to the diaphragm control unit 28. FIG. 9 shows the case in which the magnitude comparison result of the reference value Σ(K1×Z1:Kn×Zn) and the estimation value Σ(K1×A1:Kn×An) is that “the reference value Σ(K1×Z1:Kn×Zn)<the estimation value Σ(K1×A1:Kn×An).” Here, the estimation value comparison unit 27 outputs, for example, the comparison result signal=“1” representing the magnitude comparison result to the diaphragm control unit 28 as the comparison result in the frame A.

The diaphragm control unit 28 determines a direction in which the diaphragm unit 2b is driven based on the comparison result input from the estimation value comparison unit 27. The diaphragm control unit 28 controls driving of the diaphragm unit 2b in the determined direction. More specifically, if the comparison result input from the estimation value comparison unit 27 is that “the reference value Σ(K1×Z1:Kn×Zn)<the estimation value Σ(K1×A1:Kn×An),” i.e., the comparison result signal is “1,” the diaphragm control unit 28 determines that the current frame A has a larger number of focused regions of interest than the previous frame Z. Here, the diaphragm control unit 28 controls the diaphragm unit 2b to be driven by one step in the same direction in which it was previously driven. In FIG. 9, as a result of determination in the current frame A, the case in which the diaphragm unit 2b is controlled to be driven by one step in the negative direction (the opening direction) is shown.

The imaging unit 3 outputs the image signal according to the electric signal obtained by photoelectrically converting the optical image of the object of the next frame B captured through the optical system 2 to the estimation value calculation unit 25. The estimation value calculation unit 25 calculates the estimation values B1 to Bn corresponding to the regions of interest in the frame B. The estimation value calculation unit 25 outputs the one estimation value Σ(K1×B1:Kn×Bn) obtained through weighted average by multiplying the calculated estimation values B1 to Bn by the weighting coefficients K1 to Kn to the estimation value storage unit 26, the estimation value comparison unit 27, and the diaphragm control unit 28.

The estimation value comparison unit 27 replaces the above-mentioned one reference value Σ(K1×Z1:Kn×Zn) with the one estimation value Σ(K1×A1:Kn×An) of the frame A stored in the estimation value storage unit 26 in the previous frame A. The estimation value comparison unit 27 compares magnitudes of the reference value Σ(K1×A1:Kn×An) and the one estimation value Σ(K1×B1:Kn×Bn) of the frame B input from the estimation value calculation unit 25. The estimation value comparison unit 27 outputs the comparison result to the diaphragm control unit 28. FIG. 9 shows the case in which the magnitude comparison result of the reference value Σ(K1×A1:Kn×An) and the estimation value Σ(K1×B1:Kn×Bn) is that “the reference value Σ(K1×A1:Kn×An)≧the estimation value Σ(K1×B1:Kn×Bn).” Here, the estimation value comparison unit 27 outputs, for example, the comparison result signal=“0” representing the magnitude comparison result to the diaphragm control unit 28 as the comparison result in the frame B.

The diaphragm control unit 28 determines that the current frame B has a smaller number of focused regions of interest than the previous frame A if the comparison result input from the estimation value comparison unit 27 is that “the reference value Σ(K1×A1:Kn×An)≧the estimation value Σ(K1×B1:Kn×Bn),” i.e., the comparison result signal is “0.” The diaphragm control unit 28 controls the diaphragm unit 2b to be driven by one step in an opposite direction to that in which it was previously driven. In FIG. 9, as a result of determination in the current frame B, the case in which the diaphragm unit 2b is driven by one step in the positive direction (the diaphragm direction) is shown.

Thereafter, similarly, based on the determination result in the next frame, the diaphragm unit 2b is controlled to be driven. Here, the diaphragm control unit 28 controls the opening of the diaphragm unit 2b at a diaphragm position of a maximum estimation value when the maximum estimation value is detected. The diaphragm control unit 28 pauses the driving control of the diaphragm unit 2b for a preset certain time T only and holds the opening of the diaphragm unit 2b. The diaphragm control unit 28 restarts the driving control of the diaphragm unit 2b after the certain time T elapses. FIG. 9 shows the case in which the estimation value Σ(K1×C1:Kn×Cn) in the frame C is the maximum estimation value and the driving control of the diaphragm unit 2b is paused for the certain time T only.

In this way, in the endoscope system 300, the estimation value of each of the regions of interest is calculated at each of the frames imaged by the solid state imaging device 3a. As the one estimation value obtained through the weighted average of the calculated estimation values and the one estimation value (reference value) of the previous frame are compared, the opening of the diaphragm unit 2b of the next frame is controlled. Accordingly, in the endoscope system 300, like the endoscope system 100 according to the first embodiment and the endoscope system 200 according to the second embodiment, the focused image in which the opening of the diaphragm unit 2b, i.e., the range in which the object is focused on, follows a variation in the distance at which the object is distributed can be photographed.

In the endoscope system 300, when the diaphragm control unit 28 detects the maximum estimation value, the opening of the diaphragm unit 2b is controlled to be driven and held at the diaphragm position of the maximum estimation value. Accordingly, in the endoscope system 300, frequent variation of the opening of the diaphragm unit 2b, i.e., the range in which the object is focused on, due to variations such as slight noise in the electric signal of each of the frames imaged by the solid state imaging device 3a can be avoided.

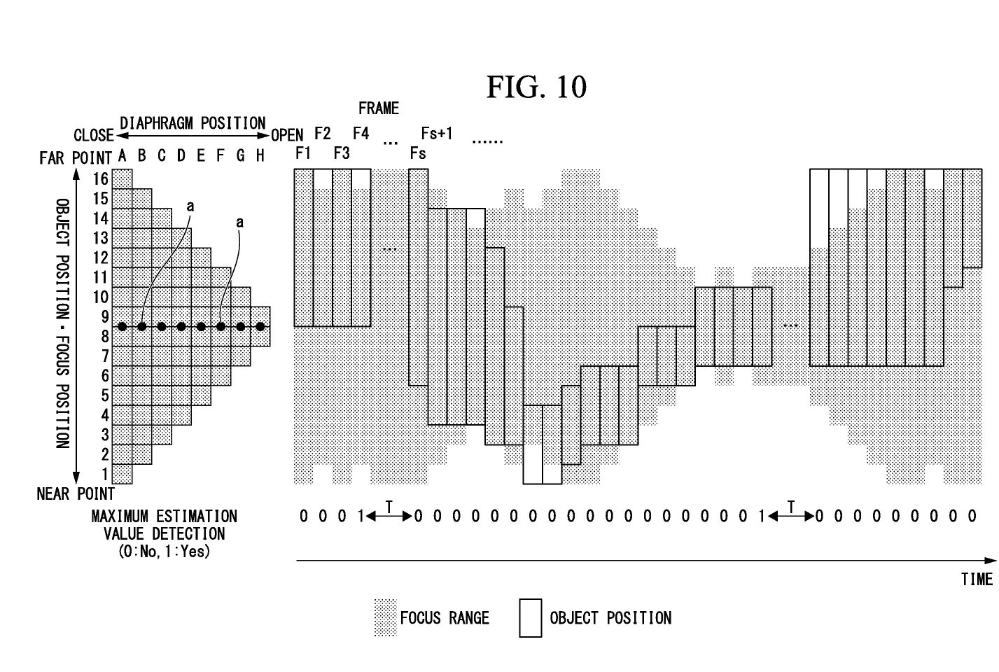

The entire control operation of the diaphragm unit 2b in the endoscope system 300 according to the third embodiment will be described. FIG. 10 is a view schematically showing an example of the entire diaphragm control operation in the endoscope system 300 according to the third embodiment. Like the entire diaphragm control operation in the endoscope system 100 according to the first embodiment shown in FIG. 4, FIG. 10 schematically shows a relation between the diaphragm position and the focus range in the optical system 2 in which the focal position is set (fixed) to a position of a center of a distance to the object serving as the photographing target and all the regions of interest is focused on when the diaphragm unit is maximally narrowed. FIG. 10 schematically shows a relation between the object position and the focus range in each of the frames when the moving object is photographed using the optical system 2 as time elapses.

A variation in the focus range by the control of the diaphragm unit 2b in the endoscope system 300 will be described using FIG. 10. Like the entire diaphragm control operation in the endoscope system 100 according to the first embodiment shown in FIG. 4, the object position and the focus range shown in FIG. 10 is a position of the object in the depth direction and a range of focusing in the depth direction.

Since a relation between the diaphragm position and the focus range in the optical system 2 of the endoscope system 300 is similar to the relation between the diaphragm position and the focus range in the endoscope system 100 according to the first embodiment shown in FIG. 4, a detailed description thereof will be omitted here.

In the endoscope system 300, the image of each of the frames is photographed using the optical system 2. According to the above-mentioned diaphragm control operation, the driving control of the diaphragm unit 2b is performed at each of the frames, and the diaphragm position when the image of the next frame is photographed is varied. In the endoscope system 300, the driving control of the diaphragm unit 2b in the opening direction and the diaphragm direction is performed twice successively, and thus the maximum estimation value can be easily detected.

FIG. 10 shows the case in which the object in the range of the object position shown by the thick-bordered box in the frame F1 (a range of a position of the object in the depth direction) is photographed while the opening of the diaphragm unit 2b is at the diaphragm position A, and based on the determination result of the diaphragm control in the frame F1, the opening of the diaphragm unit 2b upon photographing of the frame F2 is controlled to be driven by one step in the negative direction (the opening direction) at the diaphragm position B. Similarly, based on the determination result of the diaphragm control in each of the frames, the diaphragm unit 2b is controlled to be driven to the diaphragm position at which the next frame is photographed.

FIG. 10 shows the case in which the frame F4 of the object in the range of the object position shown by the thick-bordered box in the frame F4 is photographed while the opening of the diaphragm unit 2b is at the diaphragm position B, and based on the determination result of the diaphragm control in the frame F4, the opening of the diaphragm unit 2b upon photographing of the frame F5 is controlled to be driven by one step in the positive direction (the diaphragm direction) to the diaphragm position A. Here, FIG. 10 shows the case in which the estimation value of the frame F4 is detected as a maximum estimation value, and after the driving control to the diaphragm position A is performed, the driving control of the diaphragm unit 2b is paused for a certain time T only, i.e., the opening of the diaphragm unit 2b is held at the diaphragm position A of the maximum estimation value.

FIG. 10 shows the case in which, after the certain time T elapses, the object in the range of the object position shown by a thick-bordered box in a frame Fs is photographed while the opening of the diaphragm unit 2b is at the diaphragm position A, and based on the determination result of the diaphragm control in the frame Fs, the opening of the diaphragm unit 2b upon photographing of a frame Fs+1 is controlled to be driven by one step in the negative direction (the opening direction) to the diaphragm position B.

Similarly, based on the determination result and the estimation value of the diaphragm control in each of the frames, the pause or driving of the diaphragm unit 2b to the diaphragm position at which the next frame is photographed is controlled. Accordingly, as shown in FIG. 10, the diaphragm position can be controlled to follow the object position. Accordingly, in the endoscope system 300, as shown in FIG. 10, even if the object position is deviated toward a near point or a far point, the focused image in which the object is received in the focus range can be photographed.

As described above, in the endoscope system 300 according to the third embodiment, the entire image of each of the frames is divided into the plurality of regions of interest. Based on the one estimation value obtained by adding the estimation values of the divided regions of interest, the opening of the diaphragm unit 2b when the image of the next frame is imaged is controlled to follow a variation in the distance at which the object is distributed. Accordingly, like the endoscope system 100 according to the first embodiment and the endoscope system 200 according to the second embodiment, the high resolution image can be photographed at an appropriate depth of field according to the distance to the object without excessively increasing the depth of field and decreasing the resolution of the photographed image.

In the endoscope system 300 according to the third embodiment, when the maximum estimation value is detected, the diaphragm position is held. Accordingly, frequent variations of the diaphragm position, i.e., the focus range in which the object is focused on, due to variations such as slight noise in the electric signal of each of the frames can be avoided, and the image of the stable depth of field can be photographed.

In the endoscope system 300 according to the third embodiment, the case in which the diaphragm unit 2b is driven by one step at each of the frames has been described. However, the number of steps in which the diaphragm unit 2b is controlled to be driven is not limited to the above-mentioned example. For example, in consideration of the magnitudes of the reference value Σ(K1×Z1:Kn×Zn) and the estimation value Σ(K1×A1:Kn×An), the driving control of the diaphragm unit 2b may be appropriately varied, such as control of non-operation of the diaphragm unit 2b, control of driving the diaphragm unit 2b in a plurality of steps, or the like.

In the endoscope system 300 according to the third embodiment, the case in which the estimation value calculation unit 25 calculates the one estimation value obtained through weighted average of the estimation values of the regions of interest has been described. However, the method of calculating the one estimation value is not limited to the above-mentioned example. The one estimation value may be calculated through a method other than the weighted average of the estimation values of the regions of interest by the estimation value calculation unit 25. For example, like the endoscope system 100 according to the first embodiment, the estimation value calculation unit 25 may output the calculated estimation values of the regions of interest. The estimation value comparison unit 27 may compare the reference value of the previous frame and the estimation value of the current frame at each of the regions of interest.

In the endoscope system 300 according to the third embodiment, the case in which the regions of interest set by the region setting unit 24 are the plurality of regions discretely disposed with gaps and having equal sizes has been described. However, the regions of interest set by the region setting unit 24 are not limited to the above-mentioned example. For example, like the endoscope system 100 according to the first embodiment or the endoscope system 200 according to the second embodiment, various regions may be set as the regions of interest, such as in the dispositions shown in FIG. 3 or FIGS. 5A to 5D.

In the endoscope system 300 according to the third embodiment, the case in which the light source apparatus installed in the illumination unit 21 is the xenon lamp 1a and the light adjustment unit 21b configured to adjust and radiate a quantity of the light emitted from the xenon lamp 1a is included has been described. However, the light source apparatus may be a halogen lamp, an LED, a laser, or the like. The light adjustment unit 21b may not be installed in the illumination unit 21.

### Fourth Embodiment

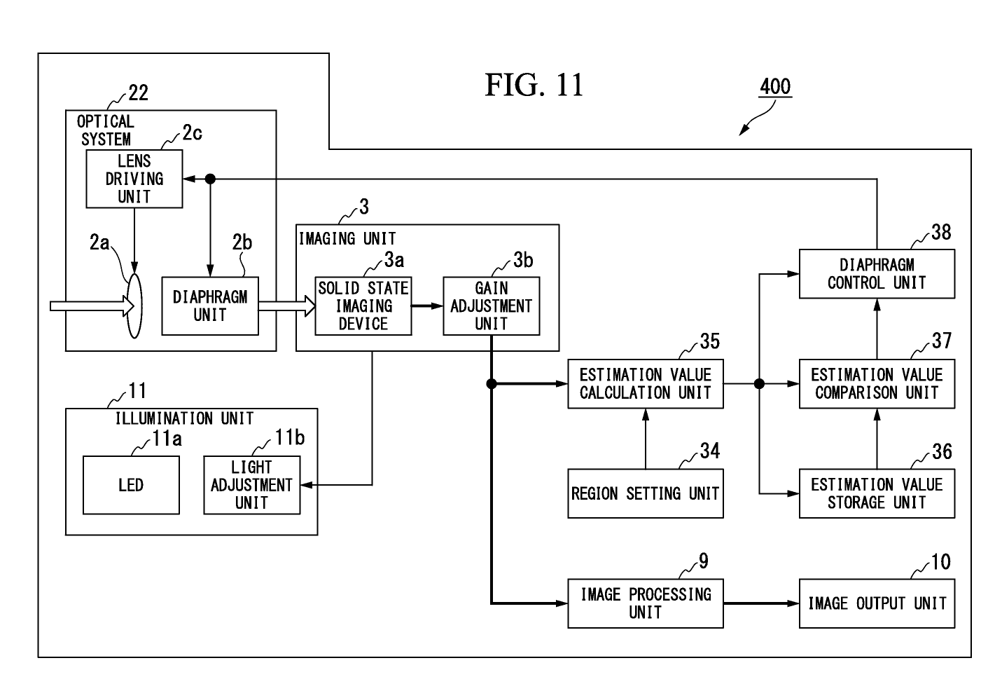

Next, an endoscope system according to a fourth embodiment will be described. FIG. 11 is a block diagram showing an example of a schematic configuration of the endoscope system according to the fourth embodiment. An endoscope system 400 shown in FIG. 11 includes an illumination unit 11, an optical system 22, an imaging unit 3, a region setting unit 34, an estimation value calculation unit 35, an estimation value storage unit 36, an estimation value comparison unit 37, and a diaphragm control unit 38. The endoscope system 400 may further include an image processing unit 9 and an image output unit 10. In the endoscope system 400 according to the fourth embodiment, the imaging unit 3, the image processing unit 9, and the image output unit 10 are the same components as those of the endoscope system 100 according to the first embodiment. The illumination unit 11 is the same component as that of the endoscope system 200 according to the second embodiment. The components of the endoscope system 400 according to the fourth embodiment different from the components of the endoscope system 100 according to the first embodiment may also include components including the same configuration as in the endoscope system 100 according to the first embodiment. Accordingly, the same components and the same configurations as the endoscope system 100 according to the first embodiment or the endoscope system 200 according to the second embodiment are designated by the same reference numerals, and a detailed description thereof will be omitted here.

The optical system 22 further includes a lens driving unit 2c in addition to the optical system 2 included in the endoscope system 100 according to the first embodiment. The lens driving unit 2c is interlocked with the opening of the diaphragm unit 2b to move the focal position of the optical lens 2a. The optical system 22 delivers the object light having a focal position varied by the lens driving unit 2c to the imaging unit 3.

The imaging unit 3 is similar to that in the endoscope system 100 according to the first embodiment. The imaging unit 3 adjusts the level of the electric signal of each of the frames imaged by the solid state imaging device 3a using the gain adjustment unit 3b. The imaging unit 3 outputs the adjusted electric signal to the estimation value calculation unit 35 and the image processing unit 9 as the image signal.

The region setting unit 34 sets the regions of interest obtained by dividing the entire image of the one frame output from the imaging unit 3 into, for example, the plurality of regions with no gap. Here, the region setting unit 34 set the magnitudes of the regions of interest to irregular sizes that reduce toward a center of the image.

The estimation value calculation unit 35 detects an amount of a high frequency element, from which a noise element is removed, from the image signal after level adjustment input from the imaging unit 3 at each of the regions of interest set by the region setting unit 34. The estimation value calculation unit 35 calculates the estimation value corresponding to the amount of the high frequency element of each of the detected regions of interest. The estimation value calculation unit 35 outputs the calculated estimation value to the estimation value storage unit 36, the estimation value comparison unit 37, and the diaphragm control unit 38.

The estimation value storage unit 36 individually stores all of the estimation values of each of the regions of interest input from the estimation value calculation unit 35 for each frame. The estimation value storage unit 36 outputs each of the stored estimation values to the estimation value comparison unit 37 as the reference value.

The estimation value comparison unit 37 reads the reference value of the corresponding region of interest stored in the estimation value storage unit 36 at the timing at which the estimation value calculation unit 35 outputs the estimation value of the current frame. The estimation value comparison unit 37 compares the estimation value of each of the regions of interest input from the estimation value calculation unit 35 and the reference value of the corresponding region of interest read from the estimation value storage unit 36 at each of the regions of interest set by the region setting unit 34.

In comparison of the estimation value and the reference value by the estimation value comparison unit 37, comparison of magnitudes of the estimation value and the reference value and calculation of an absolute value of a difference between the estimation value and the reference value (|the estimation value−the reference value|) are performed. The estimation value comparison unit 37 outputs the comparison result (the comparison result signal) obtained by comparing the magnitudes of the estimation value and the reference value and the calculation result of |the estimation value−the reference value| to the diaphragm control unit 38.

The diaphragm control unit 38 determines whether the opening of the diaphragm unit 2b installed in the optical system 2 is controlled to be driven in the diaphragm direction or the opening direction based on the calculation result and the comparison result of all the regions of interest input from the estimation value comparison unit 37. More specifically, the diaphragm control unit 38 counts the number of regions of interest for which it is determined that “the estimation value>the reference value” in the comparison result input from the estimation value comparison unit 37. The diaphragm control unit 38 determines that the diaphragm unit 2b is driven by one step in the same direction in which it was previously driven (any one of the diaphragm direction and the opening direction) if the counted result is equal to or larger than the preset number. Here, the diaphragm control unit 38 outputs a control signal for controlling driving of the diaphragm unit 2b in the determined direction. The diaphragm control unit 38 determines that the diaphragm unit 2b is driven by one step in an opposite direction to that in which it was previously driven if the counted result is smaller than the preset number. Here, the diaphragm control unit 38 outputs a control signal for controlling driving of the diaphragm unit 2b in the determined direction.

The diaphragm control unit 38 determines whether the opening of the diaphragm unit 2b is held or not based on the estimation value of each of the regions of interest in the current frame input from the estimation value calculation unit 35. Determining by the diaphragm control unit 38 whether the opening of the diaphragm unit 2b is held or not is performed based on the estimation value of all the regions of interest in the current frame input from the estimation value calculation unit 35 or the representative value obtained through weighted average of the estimation value of a predetermined number of the regions of interest. The diaphragm control unit 38 determines that the opening of the diaphragm unit 2b is held when a position of the diaphragm unit 2b at which the calculated representative value is maximized is detected. A detection method of the maximum representative value by the diaphragm control unit 38 is the same detection method of the maximum estimation value in the diaphragm control unit 28 installed in the endoscope system 300 according to the third embodiment. That is, in the diaphragm control unit 38, instead of the estimation value used to detect the maximum estimation value by the diaphragm control unit 28 installed in the endoscope system 300 according to the third embodiment, the maximum representative value is detected using the representative value. Like the diaphragm control unit 28 installed in the endoscope system 300 according to the third embodiment, the diaphragm control unit 38 controls the opening of the diaphragm unit 2b to be at the diaphragm position at which the representative value is maximized when the maximum representative value is detected. After that, the diaphragm control unit 38 pauses the driving control of the diaphragm unit 2b and holds the diaphragm position for a preset certain time T.

The diaphragm control unit 38 may restart the driving control of the diaphragm unit 2b even while the driving control of the diaphragm unit 2b at the diaphragm position is paused, i.e., even in a state in which the certain time T does not elapse. Determining by the diaphragm control unit 38 whether the driving control of the diaphragm unit 2b is restarted or not is performed based on the calculation result of all the regions of interest input from the estimation value comparison unit 37. More specifically, if the calculation result of all the regions of interest input from the estimation value comparison unit 37 does not exceed a set value P, which is preset, the diaphragm control unit 38 determines that the pause of the driving control of the diaphragm unit 2b is continued. If the calculation result exceeding the set value P, which is preset, in the calculation result of all the regions of interest input from the estimation value comparison unit 37 is at least one, the diaphragm control unit 38 determines that the driving control of the diaphragm unit 2b from the next frame is restarted, without waiting for the certain time T to elapse. The driving control of the diaphragm unit 2b after determining that the diaphragm control unit 38 restarts the driving control of the diaphragm unit 2b is the same driving control as the diaphragm unit 2b by the diaphragm control unit 28 installed in the endoscope system 300 according to the third embodiment.

The image processing unit 9 outputs image data obtained through image processing of converting the image signal of each of the frames input from the imaging unit 3 into, for example, a format displayed on a monitor connected to the endoscope system 400, to the image output unit 10. The image output unit 10 outputs and displays the image data input from the image processing unit 9 to, for example, the monitor connected to the endoscope system 400 at each of the frames.

According to the above-mentioned configuration, in the endoscope system 400, the image of the one frame imaged by the solid state imaging device 3a is divided into the plurality of regions of interest. Based on the estimation value of the divided regions of interest, the opening of the diaphragm unit 2b when the image of the next frame is imaged is controlled. Accordingly, in the endoscope system 400, like the endoscope systems according to the first to third embodiments, as the opening of the diaphragm unit 2b is controlled according to the distance at which the object is distributed, the high resolution focused image photographed by increasing the opening of the diaphragm unit 2b or the same focused image as the related art photographed by reducing the opening of the diaphragm unit 2b can be photographed.

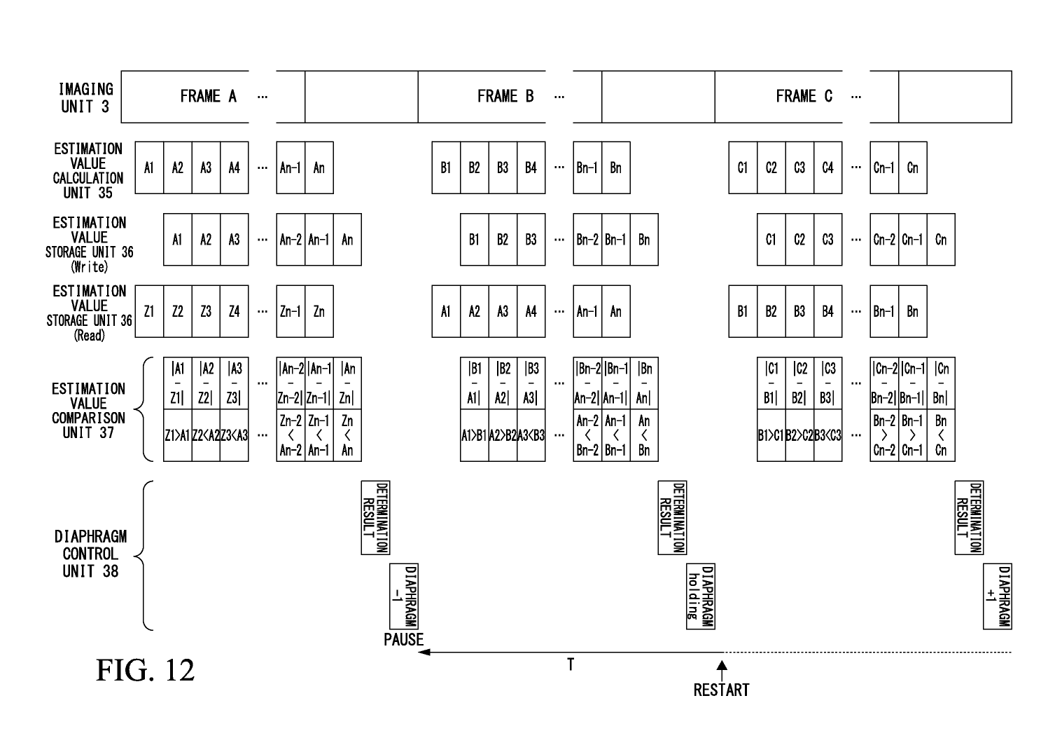

Next, an operation of the endoscope system 400 according to the fourth embodiment will be described. FIG. 12 is a timing chart showing an example of a diaphragm control operation in the endoscope system 400 according to the fourth embodiment. As described above, in the endoscope system 400, the estimation value calculation unit 35 calculates the estimation value of the region of interest at each of the frames imaged by the solid state imaging device 3a. The endoscope system 400 controls driving of the diaphragm unit 2b based on the estimation value of each of the regions of interest.

In the following description, the case in which the region setting unit 34 sets the regions of interest obtained by dividing the entire image of the one frame output from the imaging unit 3 into the plurality of regions disposed with no gap and having irregular sizes that reduce toward a center of the image will be described. In this case, the regions of interest set by the region setting unit 34 are, for example, the regions of interest shown in FIG. 5C. As the regions of interest are set as shown in FIG. 5C, in a state in which the region of the observation target in the endoscope system 400 is set as the entire image, the driving control of the diaphragm unit 2b that gives priority to the position of the center at which the object of interest is likely to be photographed can be performed.

The endoscope system 400 includes the lens driving unit 2c installed in the optical system 22. The lens driving unit 2c is interlocked with the opening of the diaphragm unit 2b to move the focal position of the optical lens 2a. The lens driving unit 2c is set to give priority to the resolution upon proximity observation in the endoscope system 400. The lens driving unit 2c moves the focal position of the optical lens 2a such that, regardless of the diaphragm position at which the opening of the diaphragm unit 2b is disposed, the object disposed at the near point is included in the focus range and the depth of field which is increased by reducing the opening of the diaphragm unit 2b can be effectively used.

In the optical system installed in the endoscope system of the related art, as the opening of the diaphragm unit is increased, the focal position of the optical lens moves toward the far point. However, in the optical system 22 of the endoscope system 400, conversely, as the opening of the diaphragm unit 2b is reduced, the focal position of the optical lens 2a moves toward the far point. That is, when the opening of the diaphragm unit 2b is reduced (driven in the diaphragm direction), the lens driving unit 2c is interlocked with movement (opening) of the diaphragm unit 2b and gradually moves the focal position of the optical lens 2a in the direction of the far point. The lens driving unit 2c becomes a pan-focus state in which the entire observation range of the endoscope system 400 is focused on when the magnitude of the opening of the diaphragm unit 2b is minimized.

The imaging unit 3 outputs the image signal according to the electric signal obtained by photoelectrically converting the optical image of the object of the current frame A captured through the optical system 22 to the estimation value calculation unit 35. In the endoscope system 400 according to the fourth embodiment, as a means for correcting the brightness of the image, which is imaged by the solid state imaging device 3a, varying depending on the opening and closing of the diaphragm unit 2b to an appropriate brightness, the light adjustment unit 11b is installed in the illumination unit 11 and the gain adjustment unit 3b is installed in the imaging unit 3. When the image of the frame A imaged by the solid state imaging device 3a is dark, first, the imaging unit 3 primarily controls the light adjustment unit 11b in the illumination unit 11 and corrects the image of the frame A to be brightened. After the light radiated from the illumination unit 11 is brightened to a maximum value, the imaging unit 3 controls amplification of the electric signal output from the solid state imaging device 3a using the gain adjustment unit 3b, and thus reduces the noise element of the image of the frame A. Accordingly, the imaging unit 3 outputs the image signal controlled to the certain brightness regardless of the opening and closing of the diaphragm unit 2b to the estimation value calculation unit 35 as the image signal of the frame A.

Here, the estimation value calculation unit 35 calculates the estimation value obtained by detecting the amount of the high frequency element, from which the noise element is removed, from the image signal input from the imaging unit 3 at each of the regions of interest set by the region setting unit 34 and obtained by dividing the entire image of the frame A into n regions disposed with no gap and having irregular sizes that reduce toward a center of the image in which the object of interest is likely to be photographed. In “A” and “1 to n” in the estimation values A1 to An shown in FIG. 12, like the description of the operation of the endoscope system 100 according to the first embodiment, “A” represents the estimation value of the frame A and “1 to n” represent the corresponding regions of interest. Also, in the following description, when the estimation value of the frame A is represented with no discrimination of the region of interest, it is referred to as “the estimation value A.” The estimation value calculation unit 35 sequentially outputs the calculated estimation values A1 to An corresponding to the regions of interest of the frame A to the estimation value storage unit 36, the estimation value comparison unit 37, and the diaphragm control unit 38.

The estimation value storage unit 36 sequentially stores the estimation values A1 to An of the frame A input from the estimation value calculation unit 35 into storage regions in the estimation value storage unit 36 corresponding to the regions of interest, respectively.

The estimation value comparison unit 37 compares the estimation value A of the frame A input from the estimation value calculation unit 35 and the reference value Z serving as the estimation value stored in the estimation value storage unit 36 and corresponding to the same region of interest as the previous frame Z. In “Z” and “1 to n” in the reference values Z1 to Zn shown in FIG. 12, like the description of the operation of the endoscope system 100 according to the first embodiment, “Z” represents the reference value of the frame Z and “1 to n” represent the corresponding regions of interest. Also, in the following description, when the reference value of the frame Z is represented with no discrimination of the region of interest, it is referred to as “the reference value Z.”

More specifically, as shown in FIG. 12, the estimation value comparison unit 37 sequentially reads the reference values Z1 to Zn of the previous frame Z of the corresponding regions of interest stored in the estimation value storage unit 36 at the timing at which the estimation value calculation unit 35 outputs the estimation values A1 to An of the frame A. The estimation value comparison unit 37 sequentially compares magnitudes of the estimation value A and the reference value Z at each of the regions of interest. The estimation value comparison unit 37 sequentially outputs the comparison result (the comparison result signal) representing whether the estimation value A is larger than the reference value Z or not to the diaphragm control unit 38. The estimation value comparison unit 37 calculates an absolute value of a difference between the estimation value A and the reference value Z (|the estimation value A−the reference value Z|). The estimation value comparison unit 37 sequentially outputs the calculation result of |the estimation value A−the reference value Z| to the diaphragm control unit 38.

Provisionally, the case in which the diaphragm unit 2b is driven by one step in the negative direction (the opening direction) from the previous frame Z to the current frame A is supposed. First, the estimation value comparison unit 37 outputs the comparison result (the comparison result signal) obtained by comparing magnitude correlations of the reference value Z1 and the estimation value A1 of the initial (first) region of interest, and the calculation result to the diaphragm control unit 38. FIG. 12 shows the case in which the magnitude comparison result of the reference value Z1 and the estimation value A1 is that “the reference value Z1>the estimation value A1.” Here, the estimation value comparison unit 37 outputs, for example, the comparison result signal=“0” representing the magnitude comparison result to the diaphragm control unit 38 as the comparison result in the first region of interest. The estimation value comparison unit 37 outputs the calculation result (|the estimation value A1−the reference value Z1|) to the diaphragm control unit 38.

Next, the estimation value comparison unit 37 outputs the comparison result (the comparison result signal) in the second region of interest obtained by comparing the magnitude correlations of the reference value Z2 and the estimation value A2 of the second region of interest, and the calculation result to the diaphragm control unit 8. FIG. 12 shows the case in which the magnitude comparison result of the reference value Z2 and the estimation value A2 is that “the reference value Z2<the estimation value A2.” Here, the estimation value comparison unit 37 outputs, for example, the comparison result signal=“1” to the diaphragm control unit 38 as the comparison result in the second region of interest. The estimation value comparison unit 37 outputs the calculation result (|the estimation value A2−the reference value Z2|) to the diaphragm control unit 38.

Subsequently, similarly, the estimation value comparison unit 37 repeats comparison of the magnitude correlations of the reference value Z and the estimation value A of each of the regions of interest. The estimation value comparison unit 37 sequentially outputs the comparison result signal=“1” if “the estimation value A>the reference value Z” is true or the comparison result signal=“0” if false to the diaphragm control unit 38 as the comparison result in each of the regions of interest. The estimation value comparison unit 37 outputs the calculation result (|the estimation value A−the reference value Z|) to the diaphragm control unit 38. In this way, the estimation value comparison unit 37 sequentially outputs the comparison result obtained by comparing the magnitude correlations of the reference value Z and the estimation value A and the calculation result obtained by calculating the absolute value of a difference between the estimation value A and the reference value Z (|the estimation value A−the reference value Z|) with respect to all the n regions of interest to the diaphragm control unit 38.

The diaphragm control unit 38 counts the number of regions of interest for which it is determined that the estimation value A of the frame A is larger than the reference value Z of the frame Z, i.e., “the estimation value A>the reference value Z,” in the comparison results sequentially input from the estimation value comparison unit 37. For example, in FIG. 12, the diaphragm control unit 38 counts the number of regions of interest in which the comparison result signal is “1.” The diaphragm control unit 38 determines a direction in which the diaphragm unit 2b is driven based on the counted result and a preset constant.

More specifically, the diaphragm control unit 38 determines that the current frame A has a larger number of focused regions of interest than the previous frame Z if the counted result is equal to or larger than a preset constant M. Here, the diaphragm control unit 38 controls the diaphragm unit 2b to be driven by one step in the same direction as the direction in which the diaphragm unit 2b was previously driven. Conversely, if the counted result is smaller than the preset constant M, the diaphragm control unit 38 determines that the current frame A has a smaller number of focused regions of interest than the previous frame Z. Here, the diaphragm control unit 38 controls the diaphragm unit 2b to be driven by one step in an opposite direction of the direction in which the diaphragm unit 2b was previously driven. An operation of the diaphragm control unit 38 is similar to that of the endoscope system 100 according to the first embodiment.

Here, the diaphragm control unit 38 detects a maximum representative value based on the representative value obtained through weighted average of all or a predetermined number of the estimation values A1 to An of the current frame A input from the estimation value calculation unit 35. The diaphragm control unit 38 controls the opening of the diaphragm unit 2b to arrive at the diaphragm position at which the representative value is maximized. After that, the diaphragm control unit 38 pauses the driving control of the diaphragm unit 2b for the preset certain time T and holds the diaphragm position. FIG. 12 shows the case in which the maximum representative value in the current frame A is detected, the diaphragm unit 2b is controlled to be driven by one step in the negative direction (the opening direction), and the driving control of the diaphragm unit 2b is paused and the diaphragm position is held for the preset certain time T.

Subsequently, the imaging unit 3 outputs the image signal according to the electric signal obtained by photoelectrically converting the optical image of the object of the next frame B captured through the optical system 22 to the estimation value calculation unit 35. The estimation value calculation unit 35 calculates the estimation values B1 to Bn corresponding to the regions of interest in the frame B. The estimation value calculation unit 35 sequentially outputs the estimation values B1 to Bn to the estimation value storage unit 36, the estimation value comparison unit 37, and the diaphragm control unit 38.

The estimation value comparison unit 37 replaces the reference values Z of the above-mentioned frame Z with the estimation values A of the frame A stored in the estimation value storage unit 36 in the previous frame A, respectively. The estimation value comparison unit 37 compares the reference value A and the estimation value B of the frame B input from the estimation value calculation unit 35. The estimation value comparison unit 37 outputs the comparison result signal and the calculation result corresponding to the regions of interest to the diaphragm control unit 38. Accordingly, the diaphragm control unit 38 controls driving of the diaphragm unit 2b based on the determination result in the frame B. However, in FIG. 12, since the driving control of the diaphragm unit 2b is paused and the diaphragm position is held, the driving control of the diaphragm unit 2b is not performed.

The diaphragm control unit 38 determines whether the driving control of the diaphragm unit 2b is restarted in the next frame B input from the estimation value calculation unit 35. As described above, the determination is performed based on the calculation result of all the regions of interest input from the estimation value comparison unit 37. FIG. 12 shows the case in which the calculation result exceeding a set value P, which is preset, in the calculation result (|the estimation value B−the reference value A|) of all the regions of interest in the next frame B is at least one, and the diaphragm control unit 38 determines that the driving control of the diaphragm unit 2b is restarted from the next frame C without waiting for the certain time T to elapse.

The estimation value comparison unit 37 uses each of the estimation values B of the frame B stored in the estimation value storage unit 36 in the previous frame B as a reference value B. The estimation value comparison unit 37 compares the reference value B and an estimation value C of the next frame C input from the estimation value calculation unit 35. The estimation value comparison unit 37 outputs the comparison result signal and the calculation result corresponding to each of the regions of interest to the diaphragm control unit 38. Accordingly, the diaphragm control unit 38 controls driving of the diaphragm unit 2b based on the determination result in the frame C. FIG. 12 shows the case in which the diaphragm unit 2b is controlled to be driven by one step in the positive direction (the diaphragm direction) based on the determination result in the frame C.

In this way, in the endoscope system 400, the estimation value of each of the regions of interest is calculated at each of the frames imaged by the solid state imaging device 3a. As the calculated estimation value and the estimation value (the reference value) of the previous frame are compared, the opening of the diaphragm unit 2b in the next frame is controlled. Accordingly, in the endoscope system 400, like the endoscope systems according to the first to third embodiments, the focused image in which the opening of the diaphragm unit 2b, i.e., the range in which the object is focused on, follows a variation in the distance at which the object is distributed can be photographed.

In the endoscope system 400, the diaphragm control unit 38 controls holding of the diaphragm position due to the pause of the driving control of the diaphragm unit 2b and restarting of the driving control of the diaphragm unit 2b based on the estimation value of all the regions of interest or the representative value obtained through weighted average of the estimation value of a predetermined number of the regions of interest. Accordingly, in the endoscope system 400, like the endoscope system 300 according to the third embodiment, frequent variations of the opening of the diaphragm unit 2b, i.e., the range in which the object is focused on, due to variations such as slight noise in the electric signal of each of the frames imaged by the solid state imaging device 3a can be avoided. In the endoscope system 400, since the driving control of the diaphragm unit 2b can be restarted even before the certain time T for which the diaphragm position is held has elapsed, even if the position of the object imaged by the solid state imaging device 3a is largely varied, the diaphragm position following the object position can be controlled at an early stage.

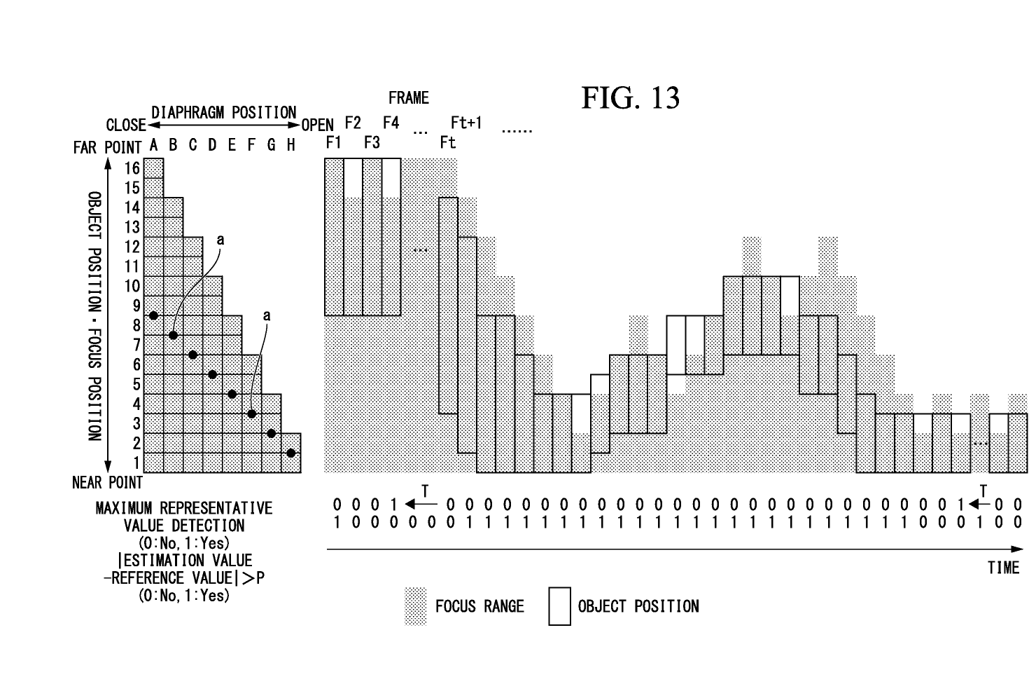

The entire control operation of the diaphragm unit 2b in the endoscope system 400 according to the fourth embodiment will be described. FIG. 13 is a view schematically showing an example of the entire diaphragm control operation in the endoscope system 400 according to the fourth embodiment. FIG. 13 schematically shows a relation between the diaphragm position and the focus range in the optical system 22 in which the focal position is interlocked with the opening of the diaphragm unit 2b to gradually move in a direction of the far point, and the range of the entire object position is focused on when the diaphragm unit is maximally narrowed. Like the entire diaphragm control operation in the endoscope system 100 according to the first embodiment shown in FIG. 4 and the entire diaphragm control operation in the endoscope system 300 according to the third embodiment shown in FIG. 10, FIG. 13 schematically shows the relation between the object position and the focus range in each of the frames when the moving object is photographed using the optical system 22 as time elapses.

A variation in the focus range by the control of the diaphragm unit 2b in the endoscope system 400 will be described using FIG. 13. Like the entire diaphragm control operation in the endoscope system 100 according to the first embodiment shown in FIG. 4 and the entire diaphragm control operation in the endoscope system 300 according to the third embodiment shown in FIG. 10, the object position and the focus range shown in FIG. 13 are also a position of the object in the depth direction and a range of focusing in the depth direction.

First, a relation between the diaphragm position and the focus range in the optical system 22 of the endoscope system 400 will be described. The optical system 22 can be controlled in eight diaphragm positions, i.e., the diaphragm positions A to H shown in FIG. 13. The focus range at each of the diaphragm positions is a range shown in FIG. 13. More specifically, the diaphragm position A, i.e., the focus range when the size of the opening of the diaphragm unit 2b is minimal, is a range of the object positions 1 to 16, i.e., a range from the near point at which the distance to the object is nearest to the far point at which the distance to the object is farthest. The focus range at the diaphragm position B is a range of the object positions 1 to 14. The focus range at the diaphragm position C is a range of the object positions 1 to 12. The focus range at the diaphragm position D is a range of the object positions 1 to 10. The focus range at the diaphragm position E is a range of the object positions 1 to 8. The focus range at the diaphragm position F is a range of the object positions 1 to 6. The focus range at the diaphragm position G is a range of the object positions 1 to 4. The diaphragm position H, i.e., the focus range when the size of the opening of the diaphragm unit 2b is maximal, is a range of the object positions 1 to 2.

As described above, the focal position of the optical system 22 is interlocked with the opening of the diaphragm unit 2b to gradually move to the position of the black spot a shown in FIG. 13. In the optical system 22, when the opening of the diaphragm unit 2b is at any one of the diaphragm positions, the nearest point is always included in the focus range.

In the endoscope system 400, the image of each of the frames is photographed using the above-mentioned optical system 22. According to the above-mentioned diaphragm control operation, the driving control of the diaphragm unit 2b is performed at each of the frames, and the diaphragm position when the image of the next frame is photographed is varied.

FIG. 13 shows the case in which the object disposed at the range of the object position (the range of the position of the object in the depth direction) shown by the thick-bordered box in the frame F1 is photographed while the opening of the diaphragm unit 2b is at the diaphragm position A, and based on the determination result of the diaphragm control in the frame F1, the opening of the diaphragm unit 2b upon photographing of the frame F2 is controlled to be driven by one step in the negative direction (the opening direction) to the diaphragm position B. Similarly, based on the determination result of the diaphragm control in each of the frames, the diaphragm unit 2b is controlled to be driven to the diaphragm position at which the next frame is photographed.

FIG. 13 shows the case in which the frame F4 of the object disposed at the range of the object position shown by the thick-bordered box in the frame F4 is photographed while the opening of the diaphragm unit 2b is at the diaphragm position B, and based on the determination result of the diaphragm control in the frame F4, the opening of the diaphragm unit 2b upon photographing of the frame F5 is controlled to be driven by one step in the positive direction (the diaphragm direction) to the diaphragm position A. Here, FIG. 13 shows the case in which the maximum representative value is detected based on the estimation value of the frame F4, and after controlling driving to the diaphragm position A, the driving control of the diaphragm unit 2b is paused for the certain time T only, i.e., the opening of the diaphragm unit 2b is held at the diaphragm position A of the maximum estimation value.

FIG. 13 shows the case in which it is determined that the driving control of the diaphragm unit 2b is restarted from the next frame Ft without waiting for the certain time T to elapse. The case in which the object disposed at the range of the object position shown by the thick-bordered box in the frame Ft is photographed while the opening of the diaphragm unit 2b is at the diaphragm position A, and based on the determination result of the diaphragm control in the frame Ft, the opening of the diaphragm unit 2b upon photographing of the frame Ft+1 is controlled to be driven by one step in the negative direction (the opening direction) at the diaphragm position B is shown.

Similarly, based on the determination result of the diaphragm control in each of the frames and the representative value obtained through weighted average of the estimation values, driving of the diaphragm unit 2b to the diaphragm position at which the next frame is photographed and pausing of the diaphragm unit 2b, and restarting of the driving control of the diaphragm unit 2b are controlled. Accordingly, as shown in FIG. 13, the diaphragm position can be controlled to follow the object position. Accordingly, in the endoscope system 400, as shown in FIG. 13, if the object position is deviated toward the near point, in a state in which the opening of the diaphragm unit 2b is increased, the focused image in which the object is received in the focus range can be photographed.

As described above, in the endoscope system 400 according to the fourth embodiment, the entire image of each of the frames is divided into the plurality of regions of interest. Based on the estimation value of the divided regions of interest, the opening of the diaphragm unit 2b when the image of the next frame is imaged is controlled to follow a variation in the distance at which the object is distributed. Accordingly, like the endoscope systems according to the first to third embodiments, a high resolution image can be photographed at an appropriate depth of field according to the distance to the object without excessively increasing the depth of field and decreasing the resolution of the photographed image.

In the endoscope system 400 according to the fourth embodiment, when the maximum representative value is detected, even before the diaphragm position has been held for the certain time T, the driving control of the diaphragm unit 2b is restarted. Accordingly, frequent variations in the diaphragm positions, i.e., the focus range in which the object is focused on, due to a variation in slight noise or the like in the electric signal of each of the frames can be avoided. In addition, if the position of the object is largely varied, the focused image that follows the object position can be photographed at an early stage.

In the endoscope system 400 according to the fourth embodiment, the case in which the lens driving unit 2c installed in the optical system 22 is interlocked with movement (opening) of the diaphragm unit 2b to gradually move the focal position of the optical lens 2a in a direction of the far point, i.e., sets a high value on the near point, has been described. However, the control method of the focal position by the lens driving unit 2c is not limited to the above-mentioned comparison method. A high value may be set for a middle near point or the far point in the control method of the focal position.

In the endoscope system 400 according to the fourth embodiment, the case in which the diaphragm unit 2b is driven by one step at each of the frames has been described. However, the number of steps in which driving of the diaphragm unit 2b is controlled is not limited to one step. For example, in consideration of the magnitudes of the reference value Z and the estimation value A, the driving control of the diaphragm unit 2b may be appropriately varied, such as control of non-operation of the diaphragm unit 2b, control of a plurality of steps of driving the diaphragm unit 2b, or the like.

In the endoscope system 400 according to the fourth embodiment, the case in which the estimation value comparison unit 37 compares the reference value of the previous frame and the estimation value of the current frame at each of the regions of interest has been described. However, the comparison method of the reference value and the estimation value in the estimation value comparison unit 37 is not limited to the above-mentioned comparison method. For example, like the endoscope system 200 according to the second embodiment or the endoscope system 300 according to the third embodiment, as the estimation value comparison unit 37 calculates statistical values, the estimation values of all the regions of interest are combined as one, and the combined one reference value and the estimation value may be compared.

In the endoscope system 400 according to the fourth embodiment, the case in which the regions of interest set by the region setting unit 34 are a plurality of regions disposed with no gap and having irregular sizes that reduce toward a center of the image has been described. However, the regions of interest set by the region setting unit 34 are not limited to the above-mentioned example. For example, like the endoscope systems according to the first to third embodiments, various regions may be set as the regions of interest, such as in the dispositions shown in FIG. 3 or FIGS. 5A to 5D.

In the endoscope system 400 according to the fourth embodiment, the case in which the light source apparatus installed in the illumination unit 11 is the LED 11a and the light adjustment unit 11b configured to adjust the light emitted from the LED 11a is included has been described. However, the light source apparatus may be a halogen lamp, a xenon lamp, a laser, or the like. The light adjustment unit 11b may not be installed in the illumination unit 11.

As described above, according to the embodiments of the present invention, the image of each of the frames is divided into a plurality of regions of interest. Based on the estimation values of the divided regions of interest, the opening of the diaphragm unit when the image of the next frame is imaged is controlled to follow a variation in the distance at which the object is distributed. Accordingly, the size of the opening of the diaphragm unit can be increased as much as possible. In addition, the image of the appropriate depth of field that follows the distance to the object can be photographed without decreasing the resolution of the photographed image.

According to the embodiments of the present invention, based on the estimation values of the regions of interest, the holding of the diaphragm position and the restarting of the driving of the diaphragm unit are controlled. Accordingly, a variation in the focus range to the object due to the slight noise or the like in the electric signal of each of the frames can be reduced. In addition, the image that follows the position of the object can be photographed at the stable depth of field.

In this way, according to the embodiments of the present invention, as the diaphragm unit is controlled, the image in the pan-focus state in which the entire observation range observed in the endoscope apparatus is focused on can be photographed.

While preferred embodiments of the invention have been described and illustrated above, it should be understood that these are exemplary of the invention and are not to be considered as limiting. Additions, omissions, substitutions, and other modifications can be made without departing from the spirit and scope of the present invention. Accordingly, the invention is not to be considered as being limited by the foregoing description, and is only limited by the scope of the appended claims.

What is claimed is:

1. An endoscope apparatus comprising:
an illumination unit configured to radiate light from a light source toward an object;
an optical system including an optical lens configured to form an object image, and a diaphragm unit configured to adjust a size of an opening in a plurality of steps;
an imaging unit including a solid state imaging device configured to convert an optical image of the object captured through the optical system into an electric signal in accordance with the optical image, the imaging unit being configured to output an image signal in accordance with an image formed based on the electric signal;
a region setting unit configured to set at least one region of interest in the image formed by the image signal output from the imaging unit;
an estimation value calculation unit configured to calculate an estimation value showing a focus level in the region of interest and output the estimation value;
an estimation value storage unit configured to store the estimation value output from the estimation value calculation unit;
an estimation value comparison unit configured to read the previous estimation value stored in the estimation value storage unit as a reference value, compare the reference value and the current estimation value output from the estimation value calculation unit, and output a comparison result of the reference value and the estimation value; and
a diaphragm control unit configured to output a control signal for controlling the opening of the diaphragm unit based on the comparison result output from the estimation value comparison unit.

2. The endoscope apparatus according to claim 1, wherein the imaging unit further includes a gain adjustment unit configured to adjust brightness of the image formed based on the electric signal by electrically adjusting the electric signal of each of frames output from the solid state imaging device, and
the imaging unit outputs the image signal in accordance with the electric signal of each of the frames adjusted such that the image has predetermined brightness.

3. The endoscope apparatus according to claim 1, wherein the illumination unit includes a light adjustment unit configured to adjust a quantity of light radiated by the light source, and
the imaging unit adjusts the quantity of light radiated by the light source by the illumination unit such that brightness of the image formed based on the electric signal at each of frames output from the solid state imaging device becomes predetermined brightness.

4. The endoscope apparatus according to claim 1, wherein the illumination unit includes a light adjustment unit configured to adjust a quantity of light radiated by the light source,
the imaging unit further includes a gain adjustment unit configured to adjust brightness of the image formed based on the electric signal by electrically adjusting the electric signal at each of frames output from the solid state imaging device, and
the imaging unit adjusts, by the gain adjustment unit, the electric signal of the frame output from the solid state imaging device after the quantity of light radiated by the light source is maximized by the illumination unit.

5. The endoscope apparatus according to claim 1, wherein the region setting unit sets the plurality of regions of interest to the entire image formed by the image signal output from the imaging unit with no gap.

6. The endoscope apparatus according to claim 1, wherein the region setting unit discretely sets the plurality of regions of interest to the image formed by the image signal output from the imaging unit with gaps.

7. The endoscope apparatus according to claim 1, wherein the region setting unit sets the regions of interest with equal sizes.

8. The endoscope apparatus according to claim 1, wherein the illumination unit includes a light adjustment unit configured to adjust a quantity of light radiated by the light source,
the imaging unit further includes a gain adjustment unit configured to adjust brightness of the image formed based on the electric signal by electrically adjusting the electric signal at each of frames output from the solid state imaging device,
the imaging unit adjusts the quantity of light radiated by the light source by the illumination unit, and outputs the image signal of each of the frames obtained by adjusting, by the gain adjustment unit, the electric signal of each of the frames output from the solid state imaging device with the light radiated by the light source having the adjusted quantity of light radiated by the light source, and
the estimation value calculation unit calculates the estimation value corresponding to the image signal of each of the frames, based on the image signal of each of the frames obtained by adjusting the quantity of light radiated by the light source and electrically adjusting the electric signal by the imaging unit.

9. The endoscope apparatus according to claim 8, wherein the estimation value calculation unit calculates the estimation value in a current frame at each of the regions of interest set by the region setting unit,
the estimation value storage unit stores the estimation value in the current frame output from the estimation value calculation unit at each of the regions of interest,
the estimation value comparison unit compares the estimation value of each of the regions of interest in the current frame output from the estimation value calculation unit and the reference value corresponding to each of the regions of interest in a previous one frame read from the estimation value storage unit, and outputs the comparison result corresponding to each of the regions of interest obtained by comparing the estimation value and the reference value,
the diaphragm control unit controls the opening of the diaphragm unit to be driven by at least one step in the same direction as a direction in which the opening of the diaphragm unit has been controlled when moved from the previous one frame to the current frame, if a number of the comparison results representing that the estimation value in the current frame is larger than the reference value in the previous one frame is equal to or larger than a preset number in the comparison results corresponding to each of the regions of interest output from the estimation value comparison unit, and
the diaphragm control unit controls the opening of the diaphragm unit to be driven by at least one step in an opposite direction to the direction in which the opening of the diaphragm unit has been controlled when moved from the previous one frame to the current frame, if the number of the comparison results representing that the estimation value in the current frame is larger than the reference value in the previous one frame is smaller than the preset number.

10. The endoscope apparatus according to claim 8, wherein the estimation value calculation unit calculates a total value obtained by adding the each estimation value corresponding to each of the regions of interest set by the region setting unit, and outputs the total value as the estimation value in a current frame,
the estimation value storage unit stores the estimation value in the current frame output from the estimation value calculation unit,
the estimation value comparison unit compares the estimation value in the current frame output from the estimation value calculation unit and the reference value in a previous one frame read from the estimation value storage unit, and outputs the comparison result obtained by comparing the estimation value and the reference value,
the diaphragm control unit controls the opening of the diaphragm unit to be driven by at least one step in the same direction as a direction in which the opening of the diaphragm unit has been controlled when moved from the previous one frame to the current frame, if the comparison result output from the estimation value comparison unit represents that the estimation value serving as the total value in the current frame is larger than the reference value serving as the total value in the previous one frame, and
the diaphragm control unit controls the opening of the diaphragm unit to be driven by at least one step in an opposite direction to the direction in which the opening of the diaphragm unit has been controlled when moved from the previous one frame to the current frame, if the comparison result represents that the estimation value serving as the total value in the current frame is equal to or smaller than the reference value serving as the total value in the previous one frame.

11. The endoscope apparatus according to claim 8, wherein the estimation value calculation unit calculates a weighted average value obtained through weighted average of the each estimation value corresponding to each of the regions of interest set by the region setting unit, and outputs the weighted average value as the estimation value in a current frame,
the estimation value storage unit stores the estimation value in the current frame output from the estimation value calculation unit,
the estimation value comparison unit compares the estimation value in the current frame output from the estimation value calculation unit and the reference value in a previous one frame read from the estimation value storage unit, and outputs the comparison result obtained by comparing the estimation value and the reference value,
the diaphragm control unit controls the opening of the diaphragm unit to be driven by at least one step in the same direction as a direction in which the opening of the diaphragm unit has been controlled when moved from a previous one frame to the current frame, if the comparison result output from the estimation value comparison unit represents that the estimation value serving as the weighted average value in the current frame is larger than the reference value serving as the weighted average value in the previous one frame, and
the diaphragm control unit controls the opening of the diaphragm unit to be driven by at least one step in an opposite direction to the direction in which the opening of the diaphragm unit has been controlled when moved from the previous one frame to the current frame, if the comparison result represents that the estimation value serving as the weighted average value in the current frame is equal to or smaller than the reference value serving as the weighted average value in the previous one frame.

12. The endoscope apparatus according to claim 1, wherein the diaphragm control unit controls the opening of the diaphragm unit to move to a position at which the estimation value is maximized if a maximum estimation value is detected from the each estimation value in the current frame corresponding to each of the regions of interest set by the region setting unit, controls the opening of the diaphragm unit such that a state of the opening of the diaphragm unit at the position is held for a preset time, and controls the opening of the diaphragm unit to be driven by at least one step in a direction in which the opening of the diaphragm unit is reduced after the preset time elapses.

13. The endoscope apparatus according to claim 12, wherein the estimation value comparison unit calculates an absolute value of a difference between the estimation value of the current frame corresponding to each of the regions of interest set by the region setting unit and the corresponding reference value in a previous one frame stored in the estimation value storage unit, and outputs the calculated result of the absolute value as a calculation result of each of the regions of interest, and
the diaphragm control unit terminates holding the state of the opening of the diaphragm unit for the preset time and restarts control of the opening of the diaphragm unit from a next frame, if the at least one calculation result in the calculation result corresponding to each of the regions of interest output from the estimation value comparison unit during the preset time exceeds a preset value.

14. The endoscope apparatus according to claim 8, wherein the optical system further includes a lens driving unit configured to be interlocked with the opening of the diaphragm unit and to set a focal position of the optical lens.

15. The endoscope apparatus according to claim 14, wherein the lens driving unit sets the focal position of the optical lens such that, when the opening of the diaphragm unit is largest, the object disposed at a proximal end is included in a focus range of the optical system and the object disposed at the proximal end to the object disposed at a distal end are gradually included in the focus range of the optical system as the opening of the diaphragm unit is reduced gradually, and when the opening of the diaphragm unit is smallest, a range in which the optical image of the object is converted into the electric signal by the solid state imaging device is entirely included in the focus range of the optical system.
